(19) United States
(12) Patent Application Publication
(10) Pub. No.: US 2020/0062858 A1
(43) Pub. Date: Feb. 27, 2020

(54) TRISPECIFIC ANTIGEN BINDING PROTEINS

(71) Applicant: CDR-LIFE AG, Schlieren-Zurich (CH)

(72) Inventors: Leonardo BORRAS, Birmensdorf (CH); Thomas SCHLEIER, Schinznach-Bad (CH); Philipp Robert RICHLE, Zürich (CH); Dominik ESCHER, Hünenberg (CH); Christian Valdemar Vinge LEISNER, Thalwil (CH); Fabian SCHEIFELE, Mägenwil - Aargau (CH)

(21) Appl. No.: 16/532,295

(22) Filed: Aug. 5, 2019

Related U.S. Application Data
(63) Continuation of application No. PCT/EP2019/055207, filed on Mar. 1, 2019.
(60) Provisional application No. 62/637,470, filed on Mar. 2, 2018.

(51) Int. Cl. C07K 16/30; C07K 16/28; A61P 35/00; G01N 33/50

(52) U.S. Cl. CPC C07K 16/30; C07K 2317/76; C07K 16/2827; C07K 16/2809; A61P 35/00; G01N 33/5011; C07K 2317/92; C07K 2317/31; C07K 2317/622; C07K 2317/35; C07K 2317/55; C07K 2317/569; C07K 2317/24; C07K 2317/565; C07K 2317/94; C07K 2317/73; C07K 2319/30; C07K 2317/74; C07K 16/2878

(57) ABSTRACT

Trispecific antigen-binding proteins including: a first binding domain capable of binding to a cell surface protein of a tumor cell; a second binding domain capable of binding to a cell surface immune checkpoint protein of the tumor cell; and a third binding domain capable of binding to a cell surface protein of an immune cell, are provided. Methods of making trispecific antigen-binding proteins are provided.

### RELATED APPLICATIONS

This application is a continuation of International Patent Application No. PCT/EP2019/055207, filed Mar. 1, 2019, which claims priority to U.S. Provisional Patent Application Ser. No. 62/637,470, filed Mar. 2, 2018, the entire disclosures of which are hereby incorporated herein by reference.

### FIELD OF THE INVENTION

This disclosure relates to compositions and methods of making trispecific antigen-binding proteins.

### BACKGROUND

Bispecific T cell engagers activate T cells through CD3 and crosslink them to tumor-expressed antigens inducing immune synapse formation and tumor cell lysis. Bispecific T cell engagers have shown therapeutic efficacy in patients with liquid tumors; however, they do not benefit all patients. Anti-tumor immunity is limited by PD-1/PD-L1 pathway-mediated immune suppression, and patients who do not benefit from existing bispecific T cell engagers may be non-responders because their T cells are anergized via the PD-1/PD-L1 pathway. The use of monoclonal antibodies that block immune checkpoint molecules, such as PD-L1, may serve to increase a baseline T-cell-specific immune response that turns the immune system against the tumor. However, a disruption in the function of immune checkpoint molecules can lead to imbalances in immunologic tolerance that results in an unchecked immune response and toxicity in patients.

Dual targeting of a tumor associated antigen (TAA) and a cancer cell surface immune checkpoint is believed to enhance the therapeutic efficacy, restrict major escape mechanisms and increase tumor-targeting selectivity, leading to reduced systemic toxicity and improved therapeutic index. Nevertheless, these strategies typically rely on reduced affinity for the immune checkpoint and high affinity for a tumor associated antigen. These strategies fail to address the issues related to expression of the TAA on normal tissues or shedding of cell surface antigen that may create an “antigen sink” that prevents therapeutic antibodies from reaching intended tumor cell targets in vivo (see, for example, Piccione et al. mAbs, 7(5): 946-956, 2015; Herrmann et al. Blood, 132(23): 2484-2494, 2018).

There is a need for multispecific antibodies having the ability to recruit more efficiently immune cells to a tumor while selectively inhibiting immune checkpoint molecules on the tumor while minimizing imbalances in immunologic tolerance and toxicity in patients.

### SUMMARY

The present invention provides trispecific antigen binding proteins with specificity to tumor antigens and an immune cell recruiting antigen.

The present invention relates to trispecific T cell engagers that bind and activate T cells through CD3, bind a tumor specific antigen, and inhibit immune checkpoint pathways. To prevent the immune system from attacking cells indiscriminately, the trispecific antigen binding proteins bind the immune checkpoint with low affinity allowing rapid dissociation from cell surface immune checkpoint proteins like PD-L1. Simultaneous binding to a tumor associated antigen and the immune checkpoint protein PD-L1 confers avidity resulting in binding to the antigens present on the tumor cell. This allows better differentiation between cells with and without the antigens predominant in tumor cells.

Furthermore, the present invention evaluated the combined role of affinity and avidity in the ability of a trispecific antigen binding protein composed of an anti-tumor associated antigen moiety with low affinity paired with an array of affinity-modulated variants of the PD-L1 to promote selective tumor-targeting under physiological conditions.

Furthermore, the present invention describes multifunctional recombinant antigen binding protein formats that enable efficient generation and development of the trispecific antigen binding proteins of the invention. These multifunctional antigen binding protein formats utilize the efficient heterodimerization properties of the heavy chain (Fd fragment) and the light chain (L) of a Fab fragment, to form a scaffold, upon which additional functions are incorporated by additional binders including but not restricted to scFv and single domain antigen binding proteins.

In one aspect of the invention, a trispecific antigen binding protein comprising: a) a first binding domain capable of binding to a cell surface protein of a tumor cell; b) a second binding domain capable of binding to a cell surface immune checkpoint protein of the tumor cell; and c) a third binding domain capable of binding to a cell surface protein of an immune cell, wherein the first binding domain binds to a cell surface protein of a tumor cell with reduced affinity to suppress binding to non-tumor cells or a soluble form of the cell surface protein, is provided.

In one aspect of the invention, a trispecific antigen binding protein comprising: a) a first binding domain capable of binding to a cell surface protein of a tumor cell; b) a second binding domain capable of binding to a cell surface immune checkpoint protein of the tumor cell; and c) a third binding domain capable of binding to a cell surface protein of an immune cell, wherein the first and second binding domains bind target antigens with reduced affinity to suppress binding to non-tumor cells, is provided.

In certain embodiments, the cell surface protein of the tumor cell is selected from the group consisting of BCMA, CD19, CD20, CD33, CD123, CEA, LMP1, LMP2, PSMA, FAP, and HER2.

In certain embodiments, the first binding domain binds BCMA on the tumor cell.

In certain embodiments, the cell surface immune checkpoint protein of the tumor cell is selected from the group consisting of CD40, CD47, CD80, CD86, GAL9, PD-L1, and PD-L2.

In certain embodiments, the second binding domain binds PD-L1 on the tumor cell.

In certain embodiments, the third binding domain binds CD3, TCRα, TCRβ, CD16, NKG2D, CD89, CD64, or CD32a on the immune cell.

In certain embodiments, the third binding domain binds to CD3 on the immune cell.

In certain embodiments, the first binding domain affinity is between about 1 nM to about 100 nM.

In certain embodiments, the second binding domain affinity is between about 1 nM to about 100 nM.

In certain embodiments, the first binding domain affinity is between about 10 nM to about 80 nM.

In certain embodiments, the second binding domain affinity is between about 10 nM to about 80 nM.

In certain embodiments, the first and second binding domain bind target antigens on the same cell to increase binding avidity.

In certain embodiments, the first binding domain comprises low affinity to the cell surface protein of the tumor cell to reduce crosslinking to healthy cells or a soluble form of the cell surface protein.

In certain embodiments, the second binding domain comprises low affinity to the cell surface immune checkpoint protein of the tumor cell to reduce crosslinking to healthy cells.

In certain embodiments, the first and second binding domain each comprise low affinity to the target antigens of the tumor cell, wherein the trispecific antigen binding protein comprises enhanced crosslinking to the tumor cell relative to crosslinking to healthy cells.

1 In certain embodiments, the first and second binding domain bind target antigens on the same cell to reduce off-target binding to healthy tissue.

In certain embodiments, the first, second, and third binding domains have reduced off-target binding.

In certain embodiments, the cell surface protein of a tumor cell is absent or has limited expression on healthy cells relative to tumor cells.

In certain embodiments, the second binding domain has low affinity to the cell surface immune checkpoint protein of the tumor cell to reduce checkpoint inhibition on healthy cells.

In certain embodiments, the first, second, and third binding domains comprise an antibody.

In certain embodiments, the first, second, and third binding domains comprise an scFv, an sdAb, or a Fab fragment.

In certain embodiments, the second binding domain is monovalent.

In certain embodiments, the third binding domain is monovalent.

In certain embodiments, the first, second, and third binding domains are joined together by one or more linkers.

In certain embodiments, the trispecific antigen binding protein has a molecular weight of about 75 kDa to about 100 kDa.

In certain embodiments, the trispecific antigen binding protein has increased serum half-life relative to an antigen binding protein with a molecular weight of ≤about 60 kDa.

In one aspect of the invention, a trispecific antigen binding protein comprising: a) a first binding domain capable of binding to a cell surface protein of a tumor cell; b) a second binding domain capable of binding to PD-L1 on the surface of the tumor cell; and c) a third binding domain capable of binding to CD3 on the surface of a T cell, wherein the first and second binding domains bind to a cell surface protein of a tumor cell and to PD-L1 with reduced affinity to suppress binding to non-tumor cells, is provided.

In certain embodiments, the cell surface protein of the tumor cell is selected from the group consisting of BCMA, CD19, CD20, CD33, CD123, CEA, LMP1, LMP2, PSMA, FAP, and HER2.

In certain embodiments, the first binding domain binds BCMA on the tumor cell.

In certain embodiments, the first binding domain affinity is between about 1 nM to about 100 nM.

In certain embodiments, the second binding domain affinity is between about 1 nM to about 100 nM.

In certain embodiments, the first binding domain affinity is between about 10 nM to about 80 nM.

In certain embodiments, the second binding domain affinity is between about 1 nM to about 80 nM.

In certain embodiments, the first and second binding domain bind target antigens on the same cell to increase binding avidity.

In certain embodiments, the first binding domain comprises low affinity to the cell surface protein of the tumor cell to reduce crosslinking to healthy cells or a soluble form of the cell surface protein.

In certain embodiments, the second binding domain comprises low affinity to PD-L1 on the surface of the tumor cell to reduce crosslinking to healthy cells.

In certain embodiments, the first and second binding domain each comprise low affinity to the target antigens of the tumor cell, wherein the trispecific antigen binding protein comprises enhanced crosslinking to the tumor cell relative to crosslinking to healthy cells.

In certain embodiments, the first and second binding domain bind target antigens on the same cell to reduce off-target binding to healthy tissue.

In certain embodiments, the first, second, and third binding domains have reduced off-target binding.

In certain embodiments, the cell surface protein of a tumor cell is absent or has limited expression on healthy cells relative to tumor cells.

In certain embodiments, the second binding domain has low affinity to PD-L1 on the surface of the tumor cell to reduce checkpoint inhibition on healthy cells.

In certain embodiments, the first, second, and third binding domains comprise an antibody.

In certain embodiments, the first, second, and third binding domains comprise an scFv, an sdAb, or a Fab fragment.

In certain embodiments, the second binding domain is monovalent.

In certain embodiments, the third binding domain is monovalent.

In certain embodiments, the first, second, and third binding domains are joined together by one or more linkers.

In certain embodiments, the trispecific antigen binding protein has a molecular weight of about 75 kDa to about 100 kDa.

In certain embodiments, the trispecific antigen binding protein has increased serum half-life relative to an antigen binding protein with a molecular weight of <about 60 kDa.

In one aspect of the invention, a trispecific antigen binding protein comprising: a) a first antibody binding domain capable of binding to a cell surface protein of a tumor cell; b) a second antibody binding domain capable of binding to a cell surface immune checkpoint protein of the tumor cell; and c) a third antibody binding domain capable of binding to a cell surface protein of an immune cell, is provided.

In one aspect of the invention, a trispecific antigen binding protein comprising two different chains, wherein: a) one chain comprises at least one heavy chain (Fd fragment) of a Fab fragment linked to at least one additional binding domain; and b) the other chain comprises at least one light chain (L) of a Fab fragment linked to at least one additional binding domain, wherein the Fab domain optionally serves as a specific heterodimerization scaffold to which the additional binding domains are optionally linked, and the binding domains have different specificities, is provided.

In certain embodiments, the additional binding domains are an scFv or an sdAb.

In certain embodiments, the trispecific binding protein comprises: i) a first binding domain capable of binding to a cell surface protein of a tumor cell; ii) a second binding domain capable of binding to a cell surface immune checkpoint protein of the tumor cell; and iii) a third binding domain capable of binding to a cell surface protein of an immune cell.

In certain embodiments, the additional binding domains are linked to the N terminus or C terminus of the heavy chain or light chain of the Fab fragment.

In one aspect of the invention, a method of treating cancer in a subject, comprising administering to the subject a therapeutically effective amount of a trispecific antigen binding protein, wherein the trispecific antigen binding protein comprises: a) a first binding domain capable of binding to a cell surface protein of a tumor cell; b) a second binding domain capable of binding to a cell surface immune checkpoint protein of the tumor cell; and c) a third binding domain capable of binding to a cell surface protein of an immune cell, wherein the first and second binding domains bind target antigens with reduced affinity to suppress binding to non-tumor cells, is provided.

In certain embodiments, the cell surface protein of the tumor cell is selected from the group consisting of BCMA, CD19, CD20, CD33, CD123, CEA, LMP1, LMP2, PSMA, FAP, and HER2.

In certain embodiments, the first binding domain binds BCMA on the tumor cell.

In certain embodiments, the cell surface immune checkpoint protein of the tumor cell is selected from the group consisting of CD40, CD47, CD80, CD86, GAL9, PD-L1, and PD-L2.

In certain embodiments, the second binding domain binds PD-L1 on the tumor cell.

In certain embodiments, the third binding domain binds CD3, TCRα, TCRβ, CD16, NKG2D, CD89, CD64, or CD32a on the immune cell.

In certain embodiments, the third binding domain binds to CD3 on the immune cell.

In certain embodiments, the cancer is selected from the group consisting of multiple myeloma, acute myeloid leukemia, acute lymphoblastic leukemia, melanoma, EBV-associated cancer, and B cell lymphoma and leukemia.

In one aspect of the invention, an ex vivo method of identifying antigen binding domains capable of binding to a cell surface protein of a tumor cell and/or a cell surface immune checkpoint protein of a tumor cell, the method comprising: a) isolating tumor cells from a patient suffering from cancer; b) contacting the tumor cells with a panel of antigen binding domains; c) determining the binding affinity for the antigen binding domains to their target antigen; and d) selecting antigen binding domains with weaker affinity relative to a control antigen binding domain, is provided.

In certain embodiments, the ex vivo method further comprises step e) wherein the selected antigen binding domain is incorporated into a trispecific antigen binding protein.

In one aspect of the invention, an ex vivo method of identifying antigen binding domains capable of one or both of binding to a cell surface protein of a tumor cell and a cell surface immune checkpoint protein of a tumor cell, the method comprising: a) isolating peripheral blood mononuclear cells (PBMCs) or bone marrow plasma cells (PCs) and autologous bone marrow infiltrating T cells from a patient suffering from cancer; b) contacting the PBMCs or PCs with a panel of trispecific antigen binding proteins, wherein a first domain of the trispecific antigen binding protein binds to CD3 on T cells and a second domain of the trispecific antigen binding protein binds to a cell surface protein of a tumor cell and/or a cell surface immune checkpoint protein of a tumor cell; c) determining drug killing of cancer cells by measuring one or more trispecific antigen binding protein effects on immune-mediated cancer cell killing; and d) selecting the trispecific antigen binding proteins based on their ability to induce immune-mediated cancer cell killing, is provided.

In certain embodiments, a trispecific antigen binding protein effect on immune-mediated cancer cell killing comprises lactate dehydrogenase (LDH) release.

In certain embodiments, a trispecific antigen binding protein effect on immune-mediated cancer cell killing comprises number of depleted target cancer cells.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 schematically depicts the interchangeable nature of the trispecific antigen binding proteins of the invention.

FIG. 2 depicts the molecular weight (kDa), concentration (mg/mL), purity (% monomer), and yield (mg/L expression culture) for eight different multispecific antigen binding constructs expressed in cell culture.

FIG. 3 depicts the purity of four different multispecific antigen binding constructs expressed in cell culture, as measured by analytical size-exclusion chromatography.

FIG. 4A-FIG. 4C depict ELISA binding data of a BCMA-PD-L1-CD3 trispecific antigen binding protein to CD3 (FIG. 4A), BCMA (FIG. 4B), and PD-L1 (FIG. 4C).

FIG. 5 depicts ELISA data of simultaneous binding of trispecific and bispecific antibodies to BCMA-CD3.

FIG. 6 depicts the ability of the CD3-binding arm of CDR1-005 to induce T cell activation. T cell proliferation was quantified for CD3+ Jurkat T cells incubated 48 hours with immobilized anti-CD3 (on plate surface). After this incubation period, WST-1 reagent was added, and the formazan dye formed was quantitated up to 5 hours.

FIG. 7 depicts that CDR1-007 induced dose dependent activation of CD3+ Jurkat T cells upon engagement of H929 myeloma cells but not in absence of cancer cells (Jurkat T cells+HEK293 cell). T cell activation was measured by IL-2 cytokine production, and phytohemagglutinin (PHA) was used as general positive control of T cell activation.

FIG. 8 depicts increased activation of T cells isolated from human peripheral blood mononuclear cells (PBMCs) after co-culture with H929 myeloma cells upon treatment with trispecific CDR1-007 compared to bispecific CDR1-008 tandem scFv BCMA/CD3. T cell activation was measured by IL-2 cytokine production.

FIG. 9A-FIG. 9B depict a head-to-head comparison of redirected T cell killing of H929 myeloma cells mediated by trispecific CDR1-007 and bispecific CDR1-008 tandem scFv BCMA/CD3 (Fig.9A) and trispecific CDR1-007 and bispecific CDR1-020 PD-L1/CD3 (FIG. 9B). Redirected T-cell killing of H929 myeloma cells was determined by lactose dehydrogenase (LDH) release assay.

FIG. 10A-FIG. 10B depict ELISA data of simultaneous binding either to BCMA-PD-L1 (FIG. 10A) or to BCMA-CD3 (FIG. 10B) of trispecific Fab-scFv molecules, where each binding site was evaluated at different positions.

FIG. 11A-FIG. 11B depict ELISA data of simultaneous binding either to BCMA-PD-L1 (FIG. 11A) or to BCMA-CD3 (FIG. 11B) of alternative trispecific formats and alternative binding sequences.

FIG. 12A-FIG. 12B depict a comparison of redirected T cell killing of H929 myeloma cells mediated trispecific Fab-scFv molecules where each binding site is evaluated at different positions (FIG. 12A) and alternative trispecific formats and alternative binding sequences. (FIG. 12B). Redirected T-cell killing of H929 myeloma cells was determined by Lactose dehydrogenase (LDH) release assay.

FIG. 13 depicts a collection of trispecific antibodies with a broad range of binding profiles to immobilized human PD-L1 as measured by ELISA using serial dilutions of the antibodies.

FIG. 14A-FIG. 14B depict a head-to-head comparison of concentration-dependent killing of H929 myeloma cells mediated by trispecific CDR1-007 and CDR1-011 (FIG. 14A) and trispecific CDR1-007 and CDR1-017 (FIG. 14B). The effector to target cells ratio used was 5:1 (T cells : H929 cells). LDH released into the cell culture media was measured after cells were incubated for 24 hours with the compounds.

FIG. 15 depicts percentages of the different cell populations in bone marrow samples of different multiple myeloma patients used for image-based ex vivo testing of trispecific antibodies.

FIG. 16A-FIG. 16C depict the ability of trispecific antibodies with different affinities for PD-L1 to avoid crosslinking T cells and normal cells, as assessed ex vivo in bone marrow tissue from multiple myeloma patients. Samples from newly-diagnosed multiple myeloma patients (FIG. 16A), relapsed multiple myeloma patients (FIG. 16B) and multi-relapsed multiple myeloma patients (FIG. 16C) were used.

FIG. 17A-FIG. 17C depict the ability of trispecific CDR1-017 compared to a bispecific control and combination of a bispecific control and an anti-PD-L1 antibody to activate T cells from the newly-diagnosed (FIG. 17A), relapsed (FIG. 17B) and multi-relapsed (FIG. 17C) multiple myeloma patients.

FIG. 18 depicts thermal stability of trispecific molecules determined by differential scanning fluorimetry (DSF).

FIG. 19A-FIG. 19C depict stability data for CDR1-007 (FIG. 19A), CDR1-011 (FIG. 19B), CDR1-017 (FIG. 19C) at high concentrations at 37° C.

FIG. 20A-FIG. 20C depict the ability of trispecific and bispecific antibodies to induce IL-2 cytokine production upon binding to human CD3+ T cells and cancer cell line H929 cells (FIG. 20A), Raji cells (FIG. 20B), and HCT116 cells (FIG. 20C).

FIG. 21A-FIG. 21B schematically depict various trispecific and bispecific antibodies (FIG. 21A) and the corresponding legend (FIG. 21B).

FIG. 22 depicts the ability of trispecific CDR1-017 redirect CD3+ T cells to the target cell population staining for CD138 or CD269, CD319. CDR1-017 is represented by filled boxes and the bispecific control, CDR1-008, is represented by empty boxes.

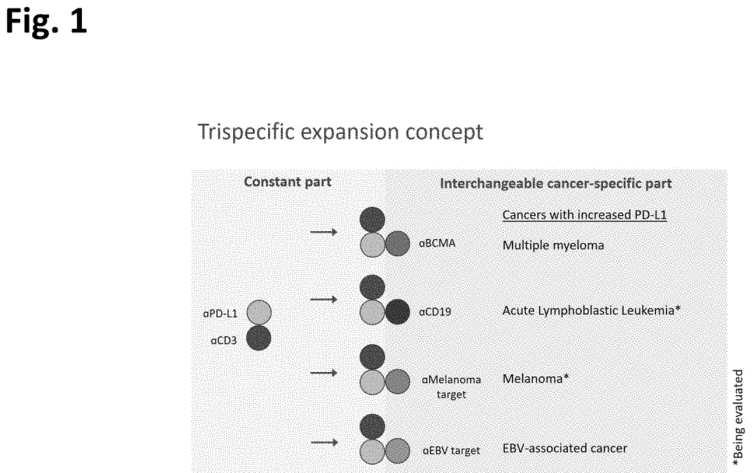

### DETAILED DESCRIPTION

Trispecific antigen binding proteins having: i) a first binding domain capable of binding to a cell surface protein of a tumor cell; ii) a second binding domain capable of binding to a cell surface immune checkpoint protein of the tumor cell; and iii) a third binding domain capable of binding to a cell surface protein of an immune cell, are provided. Methods for generating and screening trispecific antigen binding proteins are also provided. Methods for treating cancer or target tumor cell killing with the trispecific antigen binding proteins are also provided.

In certain aspects, trispecific antigen binding proteins described herein have low affinity for the tumor cell surface protein targeted by the first binding domain and low affinity for the tumor cell surface immune checkpoint protein targeted by the second binding domain. The low affinity interaction reduces the off-target binding to healthy tissue of the trispecific antigen binding proteins relative to the tumor cell or tissue.

In certain aspects, trispecific antigen binding proteins described herein have increased avidity for the tumor cell surface protein targeted by the first binding domain and for the tumor cell surface immune checkpoint protein targeted by the second binding domain. The increased avidity occurs when both cell surface proteins are present on the same cell. The increased avidity interaction reduces the off-target binding to healthy tissue of the trispecific antigen binding proteins and ensures preferential binding to the target tumor cell (see, for example, Piccione et al. mAbs, 7(5): 946-956, 2015; Kloss et al. Nature Biotechnology, 31(1): 71-75, 2013.)

Trispecific antigen binding proteins described herein are designed to be modular in nature. The trispecific antigen binding protein may comprise an unchanging core region comprising a second binding domain capable of binding to a cell surface immune checkpoint protein of a tumor cell and a third binding domain capable of binding to a cell surface protein of an immune cell. This core bispecific antigen binding protein may have an additional, first binding domain capable of binding to a cell surface protein of a tumor cell. While the core region remains unchanged, the first binding domain may be changed depending on the cancer type to be treated or tumor cell to be targeted. In an exemplary embodiment, the core region has a second binding domain capable of binding to PD-L1 on the surface of a tumor cell, and a third binding domain capable of binding CD3 on the surface of a T cell. In an exemplary embodiment, the modular first binding domain is capable of binding BCMA on the surface of a tumor cell.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

### Antigen Binding Proteins

As used herein, the term “antibody” or “antigen binding protein” refers to an immunoglobulin molecule that specifically binds to, or is immunologically reactive with an antigen or epitope, and includes both polyclonal and monoclonal antibodies, as well as functional antibody fragments, including but not limited to fragment antigen-binding (Fab) fragments, F(ab′)₂ fragments, Fab′ fragments, Fv fragments, recombinant IgG (rIgG) fragments, single chain variable fragments (scFv) and single domain antibodies (e.g., sdAb, sdFv, nanobody) fragments. The term “antibody” includes genetically engineered or otherwise modified forms of immunoglobulins, such as intrabodies, peptibodies, chimeric antibodies, fully human antibodies, humanized antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments thereof.

A Fab fragment, as used herein, is an antibody fragment comprising a light chain fragment comprising a variable light (VL) domain and a constant domain of the light chain (CL), and variable heavy (VH) domain and a first constant domain (CH1) of the heavy chain.

As used herein, the term “complementarity determining region” or “CDR” refers to non-contiguous sequences of amino acids within antibody variable regions, which confer antigen specificity and binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” or “FRs” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (AHo numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the known schemes. Likewise, unless otherwise specified, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the Kabat, Chothia, or Contact method. In other cases, the particular amino acid sequence of a CDR or FR is given.

As used herein, the term “affinity” refers to the strength of the interaction between an antibody's antigen binding site and the epitope to which it binds. As readily understood by those skilled in the art, an antibody or antigen binding protein affinity may be reported as a dissociation constant (K_{D}) in molarity (M). Many antibodies have K_{D} values in the range of 10⁻⁶ to 10⁻⁹ M. High affinity antibodies have K_{D} values of 10⁻⁹ M (1 nanomolar, nM) and lower. For example, a high affinity antibody may have K_{D} value in the range of about 1 nM to about 0.01 nM. A high affinity antibody may have K_{D} value of about 1 nM, about 0.9 nM, about 0.8 nM, about 0.7 nM, about 0.6 nM, about 0.5 nM, about 0.4 nM, about 0.3 nM, about 0.2 nM, or about 0.1 nM. Very high affinity antibodies have K_{D} values of 10⁻¹² M (1 picomolar, pM) and lower.

Low to medium affinity antibodies have KD values of greater than about 10⁻⁹M (1 nanomolar, nM). For example, a low to medium affinity antibody may have K_{D} value in the range of about 1 nM to about 100 nM. A low affinity antibody may have K_{D} value in the range of about 10 nM to about 100 nM. A low affinity antibody may have K_{D} value in the range of about 10 nM to about 80 nM. A low affinity antibody may have K_{D} value of about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, or greater than 100 nM.

The antigen binding domains of the invention may have binding affinities to their target antigen of weaker than about 10⁻⁴M, about 10⁻⁴M, about 10⁻⁵M, about 10⁻⁶ M, about 10⁻⁷ M, about 10⁻⁸M, about 10⁻⁹M, about 10⁻¹⁰ M, about 10⁻¹¹ M, about 10⁻¹² M, or about 10⁻¹³ M.

The ability of an antigen binding domain to bind to a specific antigenic determinant can be measured either through an enzyme-linked immunosorbent assay (ELISA) or other techniques familiar to one of skill in the art, e.g., surface plasmon resonance (SPR) technique (analyzed on a BlAcore instrument) (Liljeblad et al., Glyco J 17, 323-329 (2000)), and traditional binding assays (Heeley, Endocr Res 28, 217-229 (2002)).

As used herein, the term “avidity” refers to the overall strength of an antibody-antigen interaction. Avidity is the accumulated strength for multiple affinities of individual non-covalent binding interactions. As the number of simultaneous binding interactions increases, the total binding avidity increases, thus leading to a more stable interaction.

The trispecific antigen binding proteins of the invention may comprise one or more linkers for linking the domains of the trispecific antigen binding protein. The trispecific antigen binding proteins may comprise two flexible peptide linkers that covalently connect a Fab chain to two scFvs. The linkers connecting the Fab chains and the scFvs may be composed of glycine-serine (Gly-Gly-Gly-Gly-Ser) which is considered to be non-immunogenic.

Illustrative examples of linkers include glycine polymers (Gly)n; glycine-serine polymers (GlynSer)n, where n is an integer of at least one, two, three, four, five, six, seven, or eight; glycine-alanine polymers; alanine-serine polymers; and other flexible linkers known in the art.

Glycine and glycine-serine polymers are relatively unstructured, and therefore may be able to serve as a neutral tether between domains of fusion proteins such as the trispecific antigen binding proteins described herein. Glycine accesses significantly more phi-psi space than other small side chain amino acids, and is much less restricted than residues with longer side chains (Scheraga, Rev. Computational Chem. 1: 1173-142 (1992)). A person skilled in the art will recognize that design of a trispecific antigen binding protein in particular embodiments can include linkers that are all or partially flexible, such that the linker can include flexible linker stretches as well as one or more stretches that confer less flexibility to provide a desired structure.

Linker sequences can however be chosen to resemble natural linker sequences, for example, using the amino acid stretches corresponding to the beginning of human CH1 and C_{κ} sequences or amino acid stretches corresponding to the lower portion of the hinge region of human IgG.

The design of the peptide linkers connecting VL and VH domains in the scFv moieties are flexible linkers generally composed of small, non-polar or polar residues such as, e.g., Gly, Ser and Thr. A particularly exemplary linker connecting the variable domains of the scFv moieties is the (Gly₄Ser)₄ linker, where 4 is the exemplary number of repeats of the motif

Other exemplary linkers include, but are not limited to the following amino acid sequences: GGG; DGGGS; TGEKP (Liu et al, Proc. Natl. Acad. Sci.94: 5525-5530 (1997)); GGRR; (GGGGS)ₙ wherein n =1, 2, 3, 4 or 5 (Kim et al, Proc. Natl. Acad. Sci.93: 1156-1160 (1996)); EGKSSGSGSESKVD (Chaudhary et al., Proc. Natl. Acad. Sci. 87: 1066-1070 (1990)); KESGSVSSEQLAQFRSLD (Bird et al., Science 242:423-426 (1988)), GGRRGGGS; LRQRDGERP; LRQKDGGGSERP; and GSTSGSGKPGSGEGSTKG (Cooper et al, Blood, 101(4): 1637-1644 (2003)). Alternatively, flexible linkers can be rationally designed using a computer program capable of modeling the 3D structure of proteins and peptides or by phage display methods.

### Multispecific Antigen Binding Formats

In an embodiment of the invention, the trispecific antigen binding protein comprises at least one Fab domain. The Fab domain may serve as a specific heterodimerization scaffold to which additional binding domains may be linked. The natural and efficient heterodimerization properties of the heavy chain (Fd fragment) and light chain (L) of a Fab fragment makes the Fab fragment an ideal scaffold. Additional binding domains may be in several different formats, including, but not limited to, another Fab domain, a scFv, or an sdAb.

Each chain of the Fab fragment can be extended at the N- or C-terminus with additional binding domains. The chains may be co-expressed in mammalian cells, where the host-cell Binding immunoglobulin protein (BiP) chaperone drives the formation of the heavy chain-light chain heterodimer (Fd:L). These heterodimers are stable, with each of the binders retaining their specific affinities. In an exemplary embodiment for the generation of such trispecific antigen binding proteins, at least one of the above-mentioned binding sites is a Fab fragment that also serves as a specific heterodimerization scaffold. The two remaining binding sites are then fused as scFvs or sdAbs to distinct Fab chains where each chain can be extended, e.g., at the C-terminus with an additional scFv or sdAb domain (see, for example, Schoonjans et al. J. Immunology, 165(12): 7050-7057, 2000; Schoonjans et al. Biomolecular Engineering, 17: 193-202, 2001.)

Multispecific antigen binding proteins comprising two Fab domains with binding specificity to a tumor antigen and a T cell recruiting antigen (e.g., CD3) have been described (see, for example, U.S. 20150274845 A1).

An advantage of the trispecific antigen binding protein scaffolds of the invention is the intermediate molecular size of approximately 75-100 kDa. Blinatumomab, a bispecific T cell engager (BiTE), has shown excellent results in patients with relapsed or refractory acute lymphoblastic leukemia. Because of its small size (60 kDa), blinatumomab is characterized by a short serum half-life of several hours, and therefore continuous infusion is needed (see, U.S. Pat. No. 7,112,324 B1). The trispecific antigen binding proteins of the invention are expected to have significantly longer half-lives in comparison to smaller bispecific antibodies, such as BiTEs like blinatumomab, and thus, do not require continuous infusion due to their favorable half-life. An intermediate sized molecule may avoid kidney clearance and provide a half-life sufficient for improved tumor accumulation. While the trispecific antigen binding proteins of the invention have increased plasma half-life compared to other small bispecific formats, they still retain the tumor penetration ability.

An additional advantage of using Fabs as a heterodimerization unit is that Fab molecules are abundantly present in serum and therefore may be non-immunogenic when administered to a subject.

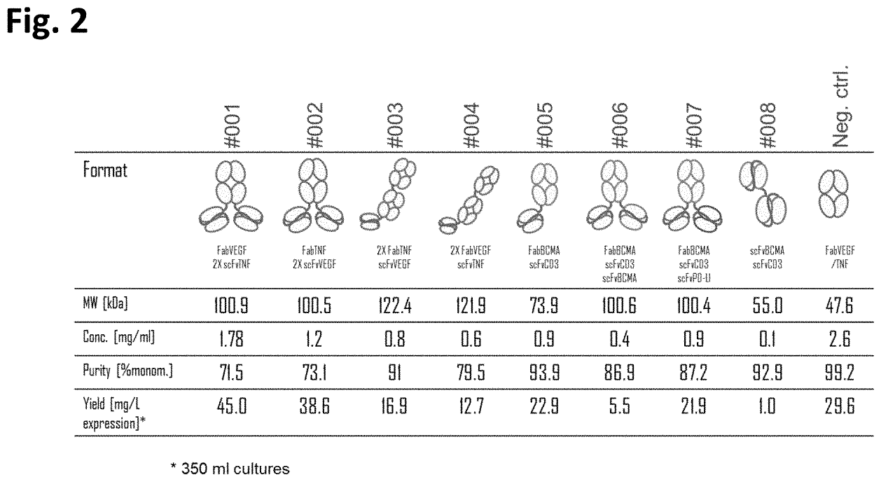

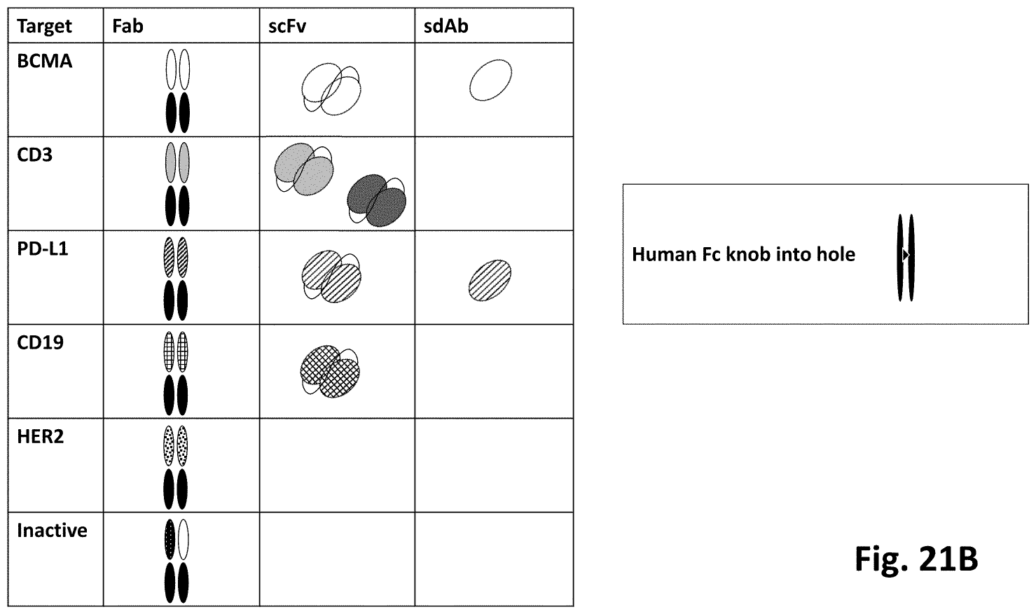

Exemplary bispecific and trispecific antigen binding protein sequences are recited below in Table 1. The sequences correspond to the antigen binding proteins of FIG. 2 and FIG. 21A-FIG. 21B.

TABLE 1
Bispecific and trispecific
antigen binding domain sequences.

| SEQ ID NO: | Sequence | Note |
|---|---|---|
| 1 | EVQLVESGGGLVQPGGSLRLSCTASG | Fd of anti-VEGF Fab |
| | FSLTDYYYMTWVRQAPGKGLEWVG | extended at the C- |
| | FIDPDDDPYYATWAKGRFTISRDNSK | terminus with anti- |
| | NTLYLQMNSLRAEDTAVYYCAGGD | TNF scFv |
| | HNSGWGLDIWGQGTLVTVSSASTKG | |
| | PSVFPLAPSSKSTSGGTAALGCLVKD | |
| | YFPEPVTVSWNSGALTSGVHTFPAVL | |
| | QSSGLYSLSSVVTVPSSSLGTQTYICN | |
| | VNHKPSNTKVDKRVEPKSCGGGGSE | |
| | IVMTQSPSTLSASVGDRVIITCQSSQS | |
| | VYGNIWMAWYQQKPGRAPKLLIYQ | |
| | ASKLASGVPSRFSGSGSGAEFTLTISS | |
| | LQPDDSATYYCQGNFNTGDRYAFGQ | |
| | GTKLTVLGGGGGSGGGGSGGGGSG | |
| | GGGSEVQLVESGGGSVQPGGSLRLS | |
| | CTASGFTISRSYWICWVRQAPGKGLE | |
| | WVGCIYGDNDITPLYANWAKGRFTI | |
| | SRDTSKNTVYLQMNSLRAEDTATYY | |
| | CARLGYADYAYDLWGQGTTVTVSS | |
| 2 | EIVMTQSPSTLSASVGDRVIITCQASEI | LC of anti-VEGF |
| | IHSWLAWYQQKPGKAPKLLIYLAST | Fab extended at the |
| | LASGVPSRFSGSGSGAEFTLTISSLQP | C-terminus with anti- |
| | DDFATYYCQNVYLASTNGANFGQGT | TNF scFv |
| | KVEIKRTVAAPSVFIFPPSDEQLKSGT | |
| | ASVVCLLNNFYPREAKVQWKVDNA | |
| | LQSGNSQESVTEQDSKDSTYSLSSTL | |
| | TLSKADYEKHKVYACEVTHQGLSSP | |
| | VTKSFNRGECGGGGSEIVMTQSPSTL | |
| | SASVGDRVIITCQSSQSVYGNIWMA | |
| | WYQQKPGRAPKLLIYQASKLASGVP | |
| | SRFSGSGSGAEFTLTISSLQPDDSATY | |
| | YCQGNFNTGDRYAFGQGTKLTVLGG | |
| | GGGSGGGGSGGGGSGGGGSEVQLVE | |
| | SGGGSVQPGGSLRLSCTASGFTISRSY | |
| | WICWVRQAPGKGLEWVGCIYGDNDI | |
| | TPLYANWAKGRFTISRDTSKNTVYL | |
| | QMNSLRAEDTATYYCARLGYADYA | |
| | YDLWGQGTTVTVSS | |
| 3 | EVQLVESGGGLVQPGGSLRLSCTASG | Fd of anti-TNF Fab |
| | FTISRSYWICWVRQAPGKGLEWVGCI | extended at the C- |
| | YGDNDITPLYANWAKGRFTISRDTSK | terminus with anti- |
| | NTVYLQMNSLRAEDTAVYYCARLG | VEGF scFv |
| | YADYAYDLWGQGTLVTVSSASTKGP | |
| | SVFPLAPSSKSTSGGTAALGCLVKDY | |
| | FPEPVTVSWNSGALTSGVHTFPAVLQ | |
| | SSGLYSLSSVVTVPSSSLGTQTYICNV | |
| | NHKPSNTKVDKRVEPKSCGGGGSEI | |
| | VMTQSPSTLSASVGDRVIITCQASEII | |
| | HSWLAWYQQKPGKAPKLLIYLASTL | |
| | ASGVPSRFSGSGSGAEFTLTISSLQPD | |
| | DSATYYCQNVYLASTNGANFGQGTK | |
| | LTVLGGGGGSGGGGSGGGGSGGGGS | |
| | EVQLVESGGGSVQPGGSLRLSCTASG | |
| | FSLTDYYYMTWVRQAPGKGLEWVG | |
| | FIDPDDDPYYATWAKGRFTISRDNSK | |
| | NTLYLQMNSLRAEDTATYYCAGGD | |
| | HNSGWGLDIWGQGTTVTVSS | |
| 4 | EIVMTQSPSTLSASVGDRVIITCQSSQ | LC of anti-TNF Fab |
| | SVYGNIWMAWYQQKPGRAPKLLIY | extended at the C- |
| | QASKLASGVPSRFSGSGSGAEFTLTIS | terminus with anti- |
| | SLQPDDFATYYCQGNFNTGDRYAFG | VEGF scFv |
| | QGTKVEIKRTVAAPSVFIFPPSDEQLK | |
| | SGTASVVCLLNNFYPREAKVQWKVD | |
| | NALQSGNSQESVTEQDSKDSTYSLSS | |
| | TLTLSKADYEKHKVYACEVTHQGLS | |
| | SPVTKSFNRGECGGGGSEIVMTQSPS | |
| | TLSASVGDRVIITCQASEIIHSWLAWY | |
| | QQKPGKAPKLLIYLASTLASGVPSRF | |
| | SGSGSGAEFTLTISSLQPDDSATYYCQ | |
| | NVYLASTNGANFGQGTKLTVLGGGG | |
| | GSGGGGSGGGGSGGGGSEVQLVESG | |
| | GGSVQPGGSLRLSCTASGFSLTDYYY | |
| | MTWVRQAPGKGLEWVGFIDPDDDP | |
| | YYATWAKGRFTISRDNSKNTLYLQM | |
| | NSLRAEDTATYYCAGGDHNSGWGL | |
| | DIWGQGTTVTVSS | |
| 5 | EVQLVESGGGLVQPGGSLRLSCTASG | Fd of anti-TNF Fab |
| | FTISRSYWICWVRQAPGKGLEWVGCI | extended at the C |
| | YGDNDITPLYANWAKGRFTISRDTSK | terminus by a second |
| | NTVYLQMNSLRAEDTAVYYCARLG | Fd of anti-TNF Fab |
| | YADYAYDLWGQGTLVTVSSASTKGP | extended at the C- |
| | SVFPLAPSSKSTSGGTAALGCLVKDY | terminus with anti- |
| | FPEPVTVSWNSGALTSGVHTFPAVLQ | VEGF scFv |
| | SSGLYSLSSVVTVPSSSLGTQTYICNV | |
| | NHKPSNTKVDKRVEPKSCGGGGSGG | |
| | GGSEVQLVESGGGLVQPGGSLRLSCT | |
| | ASGFTISRSYWICWVRQAPGKGLEW | |
| | VGCIYGDNDITPLYANWAKGRFTISR | |
| | DTSKNTVYLQMNSLRAEDTAVYYC | |
| | ARLGYADYAYDLWGQGTLVTVSSA | |
| | STKGPSVFPLAPSSKSTSGGTAALGC | |
| | LVKDYFPEPVTVSWNSGALTSGVHT | |
| | FPAVLQSSGLYSLSSVVTVPSSSLGTQ | |
| | TYICNVNHKPSNTKVDKRVEPKSCG | |
| | GGGSEIVMTQSPSTLSASVGDRVIITC | |
| | QASEIIHSWLAWYQQKPGKAPKLLIY | |
| | LASTLASGVPSRFSGSGSGAEFTLTIS | |
| | SLQPDDSATYYCQNVYLASTNGANF | |
| | GQGTKLTVLGGGGGSGGGGSGGGG | |
| | SGGGGSEVQLVESGGGSVQPGGSLR | |
| | LSCTASGFSLTDYYYMTWVRQAPGK | |
| | GLEWVGFIDPDDDPYYATWAKGRFT | |
| | ISRDNSKNTLYLQMNSLRAEDTATY | |
| | YCAGGDHNSGWGLDIWGQGTTVTV | |
| | SS | |
| 6 | EIVMTQSPSTLSASVGDRVIITCQSSQ | LC of anti-TNF Fab |
| | SVYGNIWMAWYQQKPGRAPKLLIY | |
| | QASKLASGVPSRFSGSGSGAEFTLTIS | |
| | SLQPDDFATYYCQGNFNTGDRYAFG | |
| | QGTKVEIKRTVAAPSVFIFPPSDEQLK | |
| | SGTASVVCLLNNFYPREAKVQWKVD | |
| | NALQSGNSQESVTEQDSKDSTYSLSS | |
| | TLTLSKADYEKHKVYACEVTHQGLS | |
| | SPVTKSFNRGEC | |
| 7 | EVQLVESGGGLVQPGGSLRLSCTASG | Fd of anti-VEGF Fab |
| | FSLTDYYYMTWVRQAPGKGLEWVG | extended at the C |
| | FIDPDDDPYYATWAKGRFTISRDNSK | terminus by a second |
| | NTLYLQMNSLRAEDTAVYYCAGGD | Fd of anti-VEGF Fab |
| | HNSGWGLDIWGQGTLVTVSSASTKG | extended at the C- |
| | PSVFPLAPSSKSTSGGTAALGCLVKD | terminus with anti- |
| | YFPEPVTVSWNSGALTSGVHTFPAVL | TNF scFv |
| | QSSGLYSLSSVVTVPSSSLGTQTYICN | |
| | VNHKPSNTKVDKRVEPKSCGGGGSG | |
| | GGGSEVQLVESGGGLVQPGGSLRLS | |
| | CTASGFSLTDYYYMTWVRQAPGKG | |
| | LEWVGFIDPDDDPYYATWAKGRFTI | |
| | SRDNSKNTLYLQMNSLRAEDTAVYY | |
| | CAGGDHNSGWGLDIWGQGTLVTVS | |
| | SASTKGPSVFPLAPSSKSTSGGTAAL | |
| | GCLVKDYFPEPVTVSWNSGALTSGV | |
| | HTFPAVLQSSGLYSLSSVVTVPSSSLG | |
| | TQTYICNVNHKPSNTKVDKRVEPKS | |
| | CGGGGSEIVMTQSPSTLSASVGDRVII | |
| | TCQSSQSVYGNIWMAWYQQKPGRA | |
| | PKLLIYQASKLASGVPSRFSGSGSGA | |
| | EFTLTISSLQPDDSATYYCQGNFNTG | |
| | DRYAFGQGTKLTVLGGGGGSGGGGS | |
| | GGGGSGGGGSEVQLVESGGGSVQPG | |
| | GSLRLSCTASGFTISRSYWICWVRQA | |
| | PGKGLEWVGCIYGDNDITPLYANWA | |
| | KGRFTISRDTSKNTVYLQMNSLRAED | |
| | TATYYCARLGYADYAYDLWGQGTT | |
| | VTVSS | |
| 8 | EIVMTQSPSTLSASVGDRVIITCQASEI | LC of anti-VEGF |
| | IHSWLAWYQQKPGKAPKLLIYLAST | Fab |
| | LASGVPSRFSGSGSGAEFTLTISSLQP | |
| | DDFATYYCQNVYLASTNGANFGQGT | |
| | KVEIKRTVAAPSVFIFPPSDEQLKSGT | |
| | ASVVCLLNNFYPREAKVQWKVDNA | |
| | LQSGNSQESVTEQDSKDSTYSLSSTL | |
| | TLSKADYEKHKVYACEVTHQGLSSP | |
| | VTKSFNRGEC | |
| 9 | QIQLVQSGPELKKPGETVKISCKASG | Fd of anti-BCMA |
| | YTFTDYSINWVKRAPGKGLKWMGW | Fab |
| | INTETREPAYAYDFRGRFAFSLETSAS | CDR1-005 Fd |
| | TAYLQINNLKYEDTATYFCALDYSY | |
| | AMDYWGQGTSVTVSSASTKGPSVFP | |
| | LAPSSKSTSGGTAALGCLVKDYFPEP | |
| | VTVSWNSGALTSGVHTFPAVLQSSG | |
| | LYSLSSVVTVPSSSLGTQTYICNVNH | |
| | KPSNTKVDKRVEPKSC | |
| 10 | DIVLTQSPASLAMSLGKRATISCRASE | LC of anti-BCMA |
| | SVSVIGAHLIHWYQQKPGQPPKLLIY | Fab extended at the |
| | LASNLETGVPARFSGSGSGTDFTLTID | C-terminus with anti- |
| | PVEEDDVAIYSCLQSRIFPRTFGGGTK | CD3 scFv |
| | LEIKRTVAAPSVFIFPPSDEQLKSGTA | CDR1-005 LC |
| | SVVCLLNNFYPREAKVQWKVDNAL | |
| | QSGNSQESVTEQDSKDSTYSLSSTLT | |
| | LSKADYEKHKVYACEVTHQGLSSPV | |
| | TKSFNRGECGGGGSAVVTQEPSLTVS | |
| | PGGTVTLTCGSSTGAVTTSNYANWV | |
| | QQKPGKSPRGLIGGTNKRAPGVPARF | |
| | SGSLLGGKAALTISGAQPEDEADYYC | |
| | ALWYSNHWVFGGGTKLTVLGGGGG | |
| | SGGGGSGGGGSGGGGSEVQLVESGG | |
| | GSVQPGGSLRLSCAASGFTFSTYAMN | |
| | WVRQAPGKGLEWVGRIRSKANNYA | |
| | TYYADSVKGRFTISRDDSKNTLYLQ | |
| | MNSLRAEDTATYYCVRHGNFGDSY | |
| | VSWFAYWGQGTTVTVSS | |
| 11 | QIQLVQSGPELKKPGETVKISCKASG | Fd of anti-BCMA |
| | YTFTDYSINWVKRAPGKGLKWMGW | Fab extended at the |
| | INTETREPAYAYDFRGRFAFSLETSAS | C-terminus with anti- |
| | TAYLQINNLKYEDTATYFCALDYSY | BCMA scFv |
| | AMDYWGQGTSVTVSSASTKGPSVFP | CDR1-006 Fd |
| | LAPSSKSTSGGTAALGCLVKDYFPEP | |
| | VTVSWNSGALTSGVHTFPAVLQSSG | |
| | LYSLSSVVTVPSSSLGTQTYICNVNH | |
| | KPSNTKVDKRVEPKSCGGGGSDIVLT | |
| | QSPASLAMSLGKRATISCRASESVSVI | |
| | GAHLIHWYQQKPGQPPKLLIYLASNL | |
| | ETGVPARFSGSGSGTDFTLTIDPVEED | |
| | DVAIYSCLQSRIFPRTFGGGTKLEIKG | |
| | GGGSGGGGSGGGGSGGGGSQIQLVQ | |
| | SGPELKKPGETVKISCKASGYTFTDY | |
| | SINWVKRAPGKGLKWMGWINTETR | |
| | EPAYAYDFRGRFAFSLETSASTAYLQ | |
| | INNLKYEDTATYFCALDYSYAMDY | |
| | WGQGTSVTVSS | |
| 12 | QIQLVQSGPELKKPGETVKISCKASG | Fd of anti-BCMA |
| | YTFTDYSINWVKRAPGKGLKWMGW | Fab extended at the |
| | INTETREPAYAYDFRGRFAFSLETSAS | C-terminus with anti- |
| | TAYLQINNLKYEDTATYFCALDYSY | PD-L1 scFv |
| | AMDYWGQGTSVTVSSASTKGPSVFP | CDR1-007 Fd |
| | LAPSSKSTSGGTAALGCLVKDYFPEP | |
| | VTVSWNSGALTSGVHTFPAVLQSSG | |
| | LYSLSSVVTVPSSSLGTQTYICNVNH | |
| | KPSNTKVDKRVEPKSCGGGGSEIVM | |
| | TQSPSTLSASVGDRVIITCQASEDIYS | |
| | LLAWYQQKPGKAPKLLIYDASDLAS | |
| | GVPSRFSGSGSGAEFTLTISSLQPDDS | |
| | ATYYCQGNYGSSSSSSYGAVFGQGT | |
| | KLTVLGGGGGSGGGGSGGGGSGGG | |
| | GSEVQLVESGGGSVQPGGSLRLSCTV | |
| | SGIDLSSYTMGWVRQAPGKGLEWV | |
| | GIISSGGRTYYASWAKGRFTISRDTSK | |
| | NTVYLQMNSLRAEDTATYYCARGR | |
| | YTGYPYYFALWGQGTTVTVSS | |
| 13 | DIVLTQSPASLAMSLGKRATISCRASE | scFv BCMA/scFv |
| | SVSVIGAHLIHWYQQKPGQPPKLLIY | CD3 BiTE |
| | LASNLETGVPARFSGSGSGTDFTLTID | CDR1-008 |
| | PVEEDDVAIYSCLQSRIFPRTFGGGTK | |
| | LEIKGGGGSGGGGSGGGGSGGGGSQ | |
| | IQLVQSGPELKKPGETVKISCKASGY | |
| | TFTDYSINWVKRAPGKGLKWMGWI | |
| | NTETREPAYAYDFRGRFAFSLETSAS | |
| | TAYLQINNLKYEDTATYFCALDYSY | |
| | AMDYWGQGTSVTVSSGGGGSAVVT | |
| | QEPSLTVSPGGTVTLTCGSSTGAVTT | |
| | SNYANWVQQKPGKSPRGLIGGTNKR | |
| | APGVPARFSGSLLGGKAALTISGAQP | |
| | EDEADYYCALWYSNHWVFGGGTKL | |
| | TVLGGGGGSGGGGSGGGGSGGGGSE | |
| | VQLVESGGGSVQPGGSLRLSCAASGF | |
| | TFSTYAMNWVRQAPGKGLEWVGRI | |
| | RSKANNYATYYADSVKGRFTISRDD | |
| | SKNTLYLQMNSLRAEDTATYYCVRH | |
| | GNFGDSYVSWFAYWGQGTTVTVSS | |
| 14 | EIVMTQSPSTLSASVGDRVIITCQSSQ | LC of non-binding |
| | SVYGNIWMAWYQQKPGRAPKLLIY | Fab extended at the |
| | QASKLASGVPSRFSGSGSGAEFTLTIS | C-terminus with anti- |
| | SLQPDDFATYYCQGNFNTGDRYAFG | CD3 scFv |
| | QGTKVEIKRTVAAPSVFIFPPSDEQLK | CDR1-020 |
| | SGTASVVCLLNNFYPREAKVQWKVD | |
| | NALQSGNSQESVTEQDSKDSTYSLSS | |
| | TLTLSKADYEKHKVYACEVTHQGLS | |
| | SPVTKSFNRGECGGGGSAVVTQEPSL | |
| | TVSPGGTVTLTCGSSTGAVTTSNYAN | |
| | WVQQKPGKSPRGLIGGTNKRAPGVP | |
| | ARFSGSLLGGKAALTISGAQPEDEAD | |
| | YYCALWYSNHWVFGGGTKLTVLGG | |
| | GGGSGGGGSGGGGSGGGGSEVQLVE | |
| | SGGGSVQPGGSLRLSCAASGFTFSTY | |
| | AMNWVRQAPGKGLEWVGRIRSKAN | |
| | NYATYYADSVKGRFTISRDDSKNTL | |
| | YLQMNSLRAEDTATYYCVRHGNFG | |
| | DSYVSWFAYWGQGTTVTVSS | |
| 15 | QIQLVQSGPELKKPGETVKISCKASG | Fd of anti-BCMA |
| | YTFTDYSINWVKRAPGKGLKWMGW | Fab extended at the |
| | INTETREPAYAYDFRGRFAFSLETSAS | C-terminus with anti- |
| | TAYLQINNLKYEDTATYFCALDYSY | CD3 scFv |
| | AMDYWGQGTSVTVSSASTKGPSVFP | CDR1-047 |
| | LAPSSKSTSGGTAALGCLVKDYFPEP | |
| | VTVSWNSGALTSGVHTFPAVLQSSG | |
| | LYSLSSVVTVPSSSLGTQTYICNVNH | |
| | KPSNTKVDKRVEPKSCGGGGSAVVT | |
| | QEPSLTVSPGGTVTLTCGSSTGAVTT | |
| | SNYANWVQQKPGKSPRGLIGGTNKR | |
| | APGVPARFSGSLLGGKAALTISGAQP | |
| | EDEADYYCALWYSNHWVFGGGTKL | |
| | TVLGGGGGSGGGGSGGGGSGGGGSE | |
| | VQLVESGGGSVQPGGSLRLSCAASGF | |
| | TFSTYAMNWVRQAPGKGLEWVGRI | |
| | RSKANNYATYYADSVKGRFTISRDD | |
| | SKNTLYLQMNSLRAEDTATYYCVRH | |
| | GNFGDSYVSWFAYWGQGTTVTVSS | |
| 16 | DIVLTQSPASLAMSLGKRATISCRASE | LC of anti-BCMA |
| | SVSVIGAHLIHWYQQKPGQPPKLLIY | Fab extended at the |
| | LASNLETGVPARFSGSGSGTDFTLTID | C-terminus with anti- |
| | PVEEDDVAIYSCLQSRIFPRTFGGGTK | PD-L1 scFv |
| | LEIKRTVAAPSVFIFPPSDEQLKSGTA | CDR1-047 |
| | SVVCLLNNFYPREAKVQWKVDNAL | |
| | QSGNSQESVTEQDSKDSTYSLSSTLT | |
| | LSKADYEKHKVYACEVTHQGLSSPV | |
| | TKSFNRGECGGGGSEIVMTQSPSTLS | |
| | ASVGDRVIITCQASEDIYSLLAWYQQ | |
| | KPGKAPKLLIYDASDLASGVPSRFSG | |
| | SGSGAEFTLTISSLQPDDSATYYCQG | |
| | NYGSSSSSSYGAVFGQGTKLTVLGG | |
| | GGGSGGGGSGGGGSGGGGSEVQLVE | |
| | SGGGSVQPGGSLRLSCTVSGIDLSSY | |
| | TMGWVRQAPGKGLEWVGIISSGGRT | |
| | YYASWAKGRFTISRDTSKNTVYLQM | |
| | NSLRAEDTATYYCARGRYTGYPYYF | |
| | ALWGQGTTVTVSS | |
| 17 | EVQLVESGGGSVQPGGSLRLSCAAS | Fd of anti-CD3 Fab |
| | GFTFSTYAMNWVRQAPGKGLEWVG | extended at the C- |
| | RIRSKANNYATYYADSVKGRFTISRD | terminus with anti- |
| | DSKNTLYLQMNSLRAEDTATYYCVR | BCMA scFv |
| | HGNFGDSYVSWFAYWGQGTTVTVS | CDR1-048 |
| | SASTKGPSVFPLAPSSKSTSGGTAAL | |
| | GCLVKDYFPEPVTVSWNSGALTSGV | |
| | HTFPAVLQSSGLYSLSSVVTVPSSSLG | |
| | TQTYICNVNHKPSNTKVDKRVEPKS | |
| | CGGGGSDIVLTQSPASLAMSLGKRAT | |
| | ISCRASESVSVIGAHLIHWYQQKPGQ | |
| | PPKLLIYLASNLETGVPARFSGSGSGT | |
| | DFTLTIDPVEEDDVAIYSCLQSRIFPR | |
| | TFGGGTKLEIKGGGGSGGGGSGGGG | |
| | SGGGGSQIQLVQSGPELKKPGETVKI | |
| | SCKASGYTFTDYSINWVKRAPGKGL | |
| | KWMGWINTETREPAYAYDFRGRFAF | |
| | SLETSASTAYLQINNLKYEDTATYFC | |
| | ALDYSYAMDYWGQGTSVTVSS | |
| 18 | AVVTQEPSLTVSPGGTVTLTCGSSTG | LC of anti-CD3 Fab |
| | AVTTSNYANWVQQKPGKSPRGLIGG | extended at the C- |
| | TNKRAPGVPARFSGSLLGGKAALTIS | terminus with anti- |
| | GAQPEDEADYYCALWYSNHWVFGG | PD-L1 scFv |
| | GTKLTVLGTVAAPSVFIFPPSDEQLKS | CDR1-048 |
| | GTASVVCLLNNFYPREAKVQWKVD | |
| | NALQSGNSQESVTEQDSKDSTYSLSS | |
| | TLTLSKADYEKHKVYACEVTHQGLS | |
| | SPVTKSFNRGECGGGGSEIVMTQSPS | |
| | TLSASVGDRVIITCQASEDIYSLLAW | |
| | YQQKPGKAPKLLIYDASDLASGVPSR | |
| | FSGSGSGAEFTLTISSLQPDDSATYYC | |
| | QGNYGSSSSSSYGAVFGQGTKLTVL | |
| | GGGGGSGGGGSGGGGSGGGGSEVQ | |
| | LVESGGGSVQPGGSLRLSCTVSGIDL | |
| | SSYTMGWVRQAPGKGLEWVGIISSG | |
| | GRTYYASWAKGRFTISRDTSKNTVY | |
| | LQMNSLRAEDTATYYCARGRYTGYP | |
| | YYFALWGQGTTVTVSS | |
| 19 | EVQLVESGGGSVQPGGSLRLSCTVSG | Fd of anti-PD-L1 |
| | IDLSSYTMGWVRQAPGKGLEWVGIIS | Fab extended at the |
| | SGGRTYYASWAKGRFTISRDTSKNT | C-terminus with anti- |
| | VYLQMNSLRAEDTATYYCARGRYT | CD3 scFv |
| | GYPYYFALWGQGTTVTVSSASTKGP | CDR1-049 |
| | SVFPLAPSSKSTSGGTAALGCLVKDY | |
| | FPEPVTVSWNSGALTSGVHTFPAVLQ | |
| | SSGLYSLSSVVTVPSSSLGTQTYICNV | |
| | NHKPSNTKVDKRVEPKSCGGGGSAV | |
| | VTQEPSLTVSPGGTVTLTCGSSTGAV | |
| | TTSNYANWVQQKPGKSPRGLIGGTN | |
| | KRAPGVPARFSGSLLGGKAALTISGA | |
| | QPEDEADYYCALWYSNHWVFGGGT | |
| | KLTVLGGGGGSGGGGSGGGGSGGG | |
| | GSEVQLVESGGGSVQPGGSLRLSCA | |
| | ASGFTFSTYAMNWVRQAPGKGLEW | |
| | VGRIRSKANNYATYYADSVKGRFTIS | |
| | RDDSKNTLYLQMNSLRAEDTATYYC | |
| | VRHGNFGDSYVSWFAYWGQGTTVT | |
| | VSS | |
| 20 | EIVMTQSPSTLSASVGDRVIITCQASE | LC of anti-PD-L1 |
| | DIYSLLAWYQQKPGKAPKLLIYDAS | Fab extended at the |
| | DLASGVPSRFSGSGSGAEFTLTISSLQ | C-terminus with anti- |
| | PDDSATYYCQGNYGSSSSSSYGAVF | BCMA scFv |
| | GQGTKLTVLGTVAAPSVFIFPPSDEQ | CDR1-049 |
| | LKSGTASVVCLLNNFYPREAKVQWK | |
| | VDNALQSGNSQESVTEQDSKDSTYS | |
| | LSSTLTLSKADYEKHKVYACEVTHQ | |
| | GLSSPVTKSFNRGECGGGGSDIVLTQ | |
| | SPASLAMSLGKRATISCRASESVSVIG | |
| | AHLIHWYQQKPGQPPKLLIYLASNLE | |
| | TGVPARFSGSGSGTDFTLTIDPVEED | |
| | DVAIYSCLQSRIFPRTFGGGTKLEIKG | |
| | GGGSGGGGSGGGGSGGGGSQIQLVQ | |
| | SGPELKKPGETVKISCKASGYTFTDY | |
| | SINWVKRAPGKGLKWMGWINTETR | |
| | EPAYAYDFRGRFAFSLETSASTAYLQ | |
| | INNLKYEDTATYFCALDYSYAMDY | |
| | WGQGTSVTVSS | |
| 21 | QIQLVQSGPELKKPGETVKISCKASG | Fd of anti-BCMA |
| | YTFTDYSINWVKRAPGKGLKWMGW | Fab extended at the |
| | INTETREPAYAYDFRGRFAFSLETSAS | C-terminus with anti- |
| | TAYLQINNLKYEDTATYFCALDYSY | PD-L1 sdAb |
| | AMDYWGQGTSVTVSSASTKGPSVFP | CDR1-055 |
| | LAPSSKSTSGGTAALGCLVKDYFPEP | |
| | VTVSWNSGALTSGVHTFPAVLQSSG | |
| | LYSLSSVVTVPSSSLGTQTYICNVNH | |
| | KPSNTKVDKRVEPKSCGGGGSQVQL | |
| | VESGGGLVQPGGSLRLSCAASGKMS | |
| | SRRCMAWFRQAPGKGLERVAKLLTT | |
| | SGSTYLADSVKGRFTISRDNSKNTVY | |
| | LQMNSLRAEDTAVYYCAADSFEDPT | |
| | CTLVTSSGAFQYWGQGTLVTVSS | |
| 22 | AVVTQEPSLTVSPGGTVTLTCGSSTG | scFv Anti-CD3 |
| | AVTTSNYANWVQQKPGKSPRGLIGG | extended at the C- |
| | TNKRAPGVPARFSGSLLGGKAALTIS | termianl with anti- |
| | GAQPEDEADYYCALWYSNHWVFGG | PD-L1 sdAb and |
| | GTKLTVLGGGGGSGGGGSGGGGSG | anti-BCMA sdAb |
| | GGGSEVQLVESGGGSVQPGGSLRLS | CDR-056 |
| | CAASGFTFSTYAMNWVRQAPGKGLE | |
| | WVGRIRSKANNYATYYADSVKGRFT | |
| | ISRDDSKNTLYLQMNSLRAEDTATY | |
| | YCVRHGNFGDSYVSWFAYWGQGTT | |
| | VTVSSGGGGSQVQLVESGGGLVQPG | |
| | GSLRLSCAASGKMSSRRCMAWFRQA | |
| | PGKGLERVAKLLTTSGSTYLADSVK | |
| | GRFTISRDNSKNTVYLQMNSLRAEDT | |
| | AVYYCAADSFEDPTCTLVTSSGAFQ | |
| | YWGQGTLVTVSSGGGGSQVQLVESG | |
| | GGLVQPGGSLRLSCAASGFTLDYYAI | |
| | GWFRQAPGKEREGVSCISRSDGSTYY | |
| | ADSVKGRFTISRDNAKNTVYLQMNS | |
| | LKPEDTAVYYCAAAGADCSGYLRD | |
| | YEFWGQGTLVTVSS | |
| 23 | QIQLVQSGPELKKPGETVKISCKASG | “knob” arm of a |
| | YTFTDYSINWVKRAPGKGLKWMGW | heterodimeric IgG |
| | INTETREPAYAYDFRGRFAFSLETSAS | antibody comprising |
| | TAYLQINNLKYEDTATYFCALDYSY | an anti-BCMA heavy |
| | AMDYWGQGTSVTVSSASTKGPSVFP | chain |
| | LAPSSKSTSGGTAALGCLVKDYFPEP | CDR1-0057 |
| | VTVSWNSGALTSGVHTFPAVLQSSG | |
| | LYSLSSVVTVPSSSLGTQTYICNVNH | |
| | KPSNTKVDKRVEPKSCDKTHTCPPCP | |
| | APELLGGPSVFLFPPKPKDTLMISRTP | |
| | EVTCVVVDVSHEDPEVKFNWYVDG | |
| | VEVHNAKTKPREEQYNSTYRVVSVL | |
| | TVLHQDWLNGKEYKCKVSNKALPA | |
| | PIEKTISKAKGQPREPQVYTLPPSREE | |
| | MTKNQVSLWCLVKGFYPSDIAVEWE | |
| | SNGQPENNYKTTPPVLDSDGSFFLYS | |
| | KLTVDKSRWQQGNVFSCSVMHEAL | |
| | HNHYTQKSLSLSPGK | |
| 24 | EIVMTQSPSTLSASVGDRVIITCQASE | “Hole” arm of a |
| | DIYSLLAWYQQKPGKAPKLLIYDAS | heterodimeric IgG |
| | DLASGVPSRFSGSGSGAEFTLTISSLQ | antibody comprising |
| | PDDSATYYCQGNYGSSSSSSYGAVF | an anti-PD-L1 scFv |
| | GQGTKLTVLGGGGGSGGGGSGGGG | at the N terminus of |
| | SGGGGSEVQLVESGGGSVQPGGSLR | the CH2 domain |
| | LSCTVSGIDLSSYTMGWVRQAPGKG | CDR-0057 |
| | LEWVGIISSGGRTYYASWAKGRFTIS | |
| | RDTSKNTVYLQMNSLRAEDTATYYC | |
| | ARGRYTGYPYYFALWGQGTTVTVSS | |
| | GGGGSEPKSCDKTHTCPPCPAPELLG | |
| | GPSVFLFPPKPKDTLMISRTPEVTCVV | |
| | VDVSHEDPEVKFNWYVDGVEVHNA | |
| | KTKPREEQYNSTYRVVSVLTVLHQD | |
| | WLNGKEYKCKVSNKALPAPIEKTISK | |
| | AKGQPREPQVYTLPPSREEMTKNQV | |
| | SLSCAVKGFYPSDIAVEWESNGQPEN | |
| | NYKTTPPVLDSDGSFFLVSKLTVDKS | |
| | RWQQGNVFSCSVMHEALHNHYTQK | |
| | SLSLSPGK | |
| 25 | QIQLVQSGPELKKPGETVKISCKASG | “knob” arm of a |
| | YTFTDYSINWVKRAPGKGLKWMGW | heterodimeric IgG |
| | INTETREPAYAYDFRGRFAFSLETSAS | antibody comprising |
| | TAYLQINNLKYEDTATYFCALDYSY | an anti-BCMA heavy |
| | AMDYWGQGTSVTVSSASTKGPSVFP | chain extended at the |
| | LAPSSKSTSGGTAALGCLVKDYFPEP | C-terminus with anti- |
| | VTVSWNSGALTSGVHTFPAVLQSSG | CD3 scFv |
| | LYSLSSVVTVPSSSLGTQTYICNVNH | CDR-0058 |
| | KPSNTKVDKRVEPKSCDKTHTCPPCP | |
| | APELLGGPSVFLFPPKPKDTLMISRTP | |
| | EVTCVVVDVSHEDPEVKFNWYVDG | |
| | VEVHNAKTKPREEQYNSTYRVVSVL | |
| | TVLHQDWLNGKEYKCKVSNKALPA | |
| | PIEKTISKAKGQPREPQVYTLPPSREE | |
| | MTKNQVSLWCLVKGFYPSDIAVEWE | |
| | SNGQPENNYKTTPPVLDSDGSFFLYS | |
| | KLTVDKSRWQQGNVFSCSVMHEAL | |
| | HNHYTQKSLSLSPGKGGGGSAVVTQ | |
| | EPSLTVSPGGTVTLTCGSSTGAVTTS | |
| | NYANWVQQKPGKSPRGLIGGTNKRA | |
| | PGVPARFSGSLLGGKAALTISGAQPE | |
| | DEADYYCALWYSNHWVFGGGTKLT | |
| | VLGGGGGSGGGGSGGGGSGGGGSE | |
| | VQLVESGGGSVQPGGSLRLSCAASGF | |
| | TFSTYAMNWVRQAPGKGLEWVGRI | |
| | RSKANNYATYYADSVKGRFTISRDD | |
| | SKNTLYLQMNSLRAEDTATYYCVRH | |
| | GNFGDSYVSWFAYWGQGTTVTVSS | |
| 26 | DIVLTQSPASLAMSLGKRATISCRASE | LC of the anti- |
| | SVSVIGAHLIHWYQQKPGQPPKLLIY | BCMA Knob arm |
| | LASNLETGVPARFSGSGSGTDFTLTID | CDR1-0058 |
| | PVEEDDVAIYSCLQSRIFPRTFGGGTK | |
| | VEIKRTVAAPSVFIFPPSDEQLKSGTA | |
| | SVVCLLNNFYPREAKVQWKVDNAL | |
| | QSGNSQESVTEQDSKDSTYSLSSTLT | |
| | LSKADYEKHKVYACEVTHQGLSSPV | |
| | TKSFNRGEC | |
| 27 | QVQLQQSGAELVRPGSSVKISCKASG | Fd of anti-CD19 Fab |
| | YAFSSYWMNWVKQRPGQGLEWIGQ | extended at the C- |
| | IWPGDGDTNYNGKFKGKATLTADES | terminus with anti- |
| | SSTAYMQLSSLASEDSAVYFCARRET | PD-L1 scFv |
| | TTVGRYYYAMDYWGQGTTVTVSSA | CDR1-061 |
| | STKGPSVFPLAPSSKSTSGGTAALGC | |
| | LVKDYFPEPVTVSWNSGALTSGVHT | |
| | FPAVLQSSGLYSLSSVVTVPSSSLGTQ | |
| | TYICNVNHKPSNTKVDKRVEPKSCG | |
| | GGGSEIVMTQSPSTLSASVGDRVIITC | |
| | QASEDIYSLLAWYQQKPGKAPKLLIY | |
| | DASDLASGVPSRFSGSGSGAEFTLTIS | |
| | SLQPDDSATYYCQGNYGSSSSSSYGA | |
| | VFGQGTKLTVLGGGGGSGGGGSGG | |
| | GGSGGGGSEVQLVESGGGSVQPGGS | |
| | LRLSCTVSGIDLSSYTMGWVRQAPG | |
| | KGLEWVGIISSGGRTYYASWAKGRF | |
| | TISRDTSKNTVYLQMNSLRAEDTATY | |
| | YCARGRYTGYPYYFALWGQGTTVT | |
| | VSS | |
| 28 | DIQLTQSPASLAVSLGQRATISCKASQ | LC of anti-CD19 Fab |
| | SVDYDGDSYLNWYQQIPGQPPKLLIY | extended at the C- |
| | DASNLVSGIPPRFSGSGSGTDFTLNIH | terminus with anti- |
| | PVEKVDAATYHCQQSTEDPWTFGGG | CD3 scFv |
| | TKLEIKTVAAPSVFIFPPSDEQLKSGT | CDR1-061 |
| | ASVVCLLNNFYPREAKVQWKVDNA | |
| | LQSGNSQESVTEQDSKDSTYSLSSTL | |
| | TLSKADYEKHKVYACEVTHQGLSSP | |
| | VTKSFNRGECGGGGSAVVTQEPSLT | |
| | VSPGGTVTLTCGSSTGAVTTSNYAN | |
| | WVQQKPGKSPRGLIGGTNKRAPGVP | |
| | ARFSGSLLGGKAALTISGAQPEDEAD | |
| | YYCALWYSNHWVFGGGTKLTVLGG | |
| | GGGSGGGGSGGGGSGGGGSEVQLVE | |
| | SGGGSVQPGGSLRLSCAASGFTFSTY | |
| | AMNWVRQAPGKGLEWVGRIRSKAN | |
| | NYATYYADSVKGRFTISRDDSKNTL | |
| | YLQMNSLRAEDTATYYCVRHGNFG | |
| | DSYVSWFAYWGQGTTVTVSS | |
| 29 | DIQLTQSPASLAVSLGQRATISCKASQ | scFv CD19/scFv |
| | SVDYDGDSYLNWYQQIPGQPPKLLIY | CD3 BiTE |
| | DASNLVSGIPPRFSGSGSGTDFTLNIH | CDR1-063 |
| | PVEKVDAATYHCQQSTEDPWTFGGG | |
| | TKLEIKGGGGSGGGGSGGGGSGGGG | |
| | SQVQLQQSGAELVRPGSSVKISCKAS | |
| | GYAFSSYWMNWVKQRPGQGLEWIG | |
| | QIWPGDGDTNYNGKFKGKATLTADE | |
| | SSSTAYMQLSSLASEDSAVYFCARRE | |
| | TTTVGRYYYAMDYWGQGTTVTVSS | |
| | GGGGSEVQLVESGGGSVQPGGSLRL | |
| | SCAASGFTFSTYAMNWVRQAPGKGL | |
| | EWVGRIRSKANNYATYYADSVKGRF | |
| | TISRDDSKNTLYLQMNSLRAEDTATY | |
| | YCVRHGNFGDSYVSWFAYWGQGTT | |
| | VTVSSGGGGSGGGGSGGGGSGGGGS | |
| | AVVTQEPSLTVSPGGTVTLTCGSSTG | |
| | AVTTSNYANWVQQKPGKSPRGLIGG | |
| | TNKRAPGVPARFSGSLLGGKAALTIS | |
| | GAQPEDEADYYCALWYSNHWVFGG | |
| | GTKLTVLG | |
| 30 | EVQLVESGGGLVQPGGSLRLSCAAS | Fd of anti-Her2 Fab |
| | GFNIKDTYIHWVRQAPGKGLEWVAR | extended at the C- |
| | IYPTNGYTRYADSVKGRFTISADTSK | terminus with anti- |
| | NTAYLQMNSLRAEDTAVYYCSRWG | PD-L1 scFv |
| | GDGFYAMDYWGQGTLVTVSSASTK | CDR1-081 |
| | GPSVFPLAPSSKSTSGGTAALGCLVK | |
| | DYFPEPVTVSWNSGALTSGVHTFPA | |
| | VLQSSGLYSLSSVVTVPSSSLGTQTYI | |
| | CNVNHKPSNTKVDKRVEPKSCGGGG | |
| | SEIVMTQSPSTLSASVGDRVIITCQAS | |
| | EDIYSLLAWYQQKPGKAPKLLIYDAS | |
| | DLASGVPSRFSGSGSGAEFTLTISSLQ | |
| | PDDSATYYCQGNYGSSSSSSYGAVF | |
| | GQGTKLTVLGGGGGSGGGGSGGGG | |
| | SGGGGSEVQLVESGGGSVQPGGSLR | |
| | LSCTVSGIDLSSYTMGWVRQAPGKG | |
| | LEWVGIISSGGRTYYASWAKGRFTIS | |
| | RDTSKNTVYLQMNSLRAEDTATYYC | |
| | ARGRYTGYPYYFALWGQGTTVTVSS | |
| 31 | DIQMTQSPSSLSASVGDRVTITCRAS | LC of anti-Her2 Fab |
| | QDVNTAVAWYQQKPGKAPKLLIYSA | extended at the C- |
| | SFLYSGVPSRFSGSRSGTDFTLTISSLQ | terminus with anti- |
| | PEDFATYYCQQHYTTPPTFGQGTKV | CD3 scFv |
| | EIKRTVAAPSVFIFPPSDEQLKSGTAS | CDR1-082 |
| | VVCLLNNFYPREAKVQWKVDNALQ | |
| | SGNSQESVTEQDSKDSTYSLSSTLTLS | |
| | KADYEKHKVYACEVTHQGLSSPVTK | |
| | SFNRGECGGGGSAVVTQEPSLTVSPG | |
| | GTVTLTCGSSTGAVTTSNYANWVQQ | |
| | KPGKSPRGLIGGTNKRAPGVPARFSG | |
| | SLLGGKAALTISGAQPEDEADYYCAL | |
| | WYSNHWVFGGGTKLTVLGGGGGSG | |
| | GGGSGGGGSGGGGSEVQLVESGGGS | |
| | VQPGGSLRLSCAASGFTFSTYAMNW | |
| | VRQAPGKGLEWVGRIRSKANNYATY | |
| | YADSVKGRFTISRDDSKNTLYLQMN | |
| | SLRAEDTATYYCVRHGNFGDSYVSW | |
| | FAYWGQGTTVTVSS | |
| 32 | EVQLVESGGGLVQPGGSLRLSCAAS | Fd of anti-Her2 Fab |
| | GFNIKDTYIHWVRQAPGKGLEWVAR | CDR-083 |
| | IYPTNGYTRYADSVKGRFTISADTSK | |
| | NTAYLQMNSLRAEDTAVYYCSRWG | |
| | GDGFYAMDYWGQGTLVTVSSASTK | |
| | GPSVFPLAPSSKSTSGGTAALGCLVK | |
| | DYFPEPVTVSWNSGALTSGVHTFPA | |
| | VLQSSGLYSLSSVVTVPSSSLGTQTYI | |
| | CNVNHKPSNTKVDKRVEPKSC | |

Additional exemplary trispecific formats may be used as well. For example, the Tri-specific T Cell-Activating Construct (TriTAC) format may be employed. The TriTAC format comprises a mixture of scFv, sdAb, and Fab domains, although all three domains may not be employed in one antibody molecule. The TriTAC format antibody may comprise at least one half-life extension domain, e.g., a human serum albumin binding domain. Examples of the TriTAC format and exemplary TriTAC antibodies are described further in WO2016187594 and WO2018071777A1, incorporated herein by reference.

### Binding Domains to Cell Surface Proteins of Tumor Cells

Trispecific antigen binding proteins having a first binding domain capable of binding to a cell surface protein of the tumor cell are provided. The first binding domain of the trispecific antigen binding proteins is capable of inhibiting the activity of the cell surface protein and serves as a means of recruiting an immune cell specifically to the tumor cell. Examples of cell surface proteins on tumor cells that may be targeted include, but are not limited to, BCMA, CD19, CD20, CD33, CD123, CEA, LMP1, LMP2, PSMA, FAP, and HER2. An exemplary tumor cell protein is BCMA.

Examples of bispecific antigen binding proteins with binding specificity to a cell surface protein on a tumor cell includes, U.S. 20130273055 A1, U.S. Pat. No. 9,150,664 B2, U.S. 20150368351 A1, U.S. 20170218077 A1, Hipp et al. (Leukemia, 31: 1743-1751 (2017)), and Seckinger et al. (Cancer Cell, 31(3): 396-410 (2017)).

The binding affinity of the first binding domain of the trispecific antigen binding protein may be low to reduce off-target binding of the trispecific antigen binding protein to non-tumor or healthy tissue. The binding affinity of the first binding domain may be in the range of about 1 nM to about 100 nM. The binding affinity of the first binding domain may be in the range of about 1 nM to about 80 nM. The binding affinity of the first binding domain may be in the range of about 10 nM to about 80 nM.

BCMA antigen binding domain sequences are recited below in Table 2 and in WO2016094304 and WO2010104949 as an example of binding domains capable of binding a cell surface protein on a tumor cell. The sequences may be used in either a Fab, scFv, or sdAb format as part of the trispecific antigen binding protein.

TABLE 2
BCMA antigen binding domain sequences.

| SEQ | | |
|---|---|---|
| ID NO: | Sequence | Note |
| 33 | QIQLVQSGPELKKPGETV | Anti-BCMA C11 D |
| | KISCKASGYTFTDYSINW | 5.3 VH sequence |
| | VKRAPGKGLKWMGWINTE | |
| | TREPAYAYDFRGRFAFSL | |
| | ETSASTAYLQINNLKYED | |
| | TATYFCALDYSYAMDYWG | |
| | QGTSVTVSS | |
| 34 | DIVLTQSPASLAMSLGKR | Anti-BCMA C11 D |
| | ATISCRASESVSVIGAHL | 5.3 VL sequence |
| | IHWYQQKPGQPPKLLIYL | |
| | ASNLETGVPARFSGSGSG | |
| | TDFTLTIDPVEEDDVAIY | |
| | SCLQSRIFPRTFGGGTKL | |
| | EIK | |

### Binding Domains to Cell Surface Immune Checkpoint Proteins of Tumor Cells

Trispecific antigen binding proteins having a second binding domain capable of binding to a cell surface immune checkpoint protein of the tumor cell are provided. The second binding domain of the trispecific antigen binding proteins is capable of inhibiting the activity of the cell surface immune checkpoint protein, thereby inhibiting the immune-suppressive signal of the target tumor cells to be eliminated. Examples of cell surface immune checkpoint proteins on tumor cells that may be targeted include, but are not limited to, CD40, CD47, CD80, CD86, GALS, PD-L1, and PD-L2. An exemplary immune checkpoint protein is PD-L1.

In an exemplary embodiment, the trispecific antigen binding protein of the invention binds PD-L1 on the cell surface of tumor cells. Programmed death receptor 1 is an inhibitory receptor that is induced on activated T cells and expressed on exhausted T cells. PD1-PD-L1 interactions may be at least partly responsible for the state of immune dysfunction and also implicated in reduced BiTE efficacy in acute lymphoblastic leukemia patients with increased levels of PD-L1 who do not benefit from blinatumomab therapy (Krupka et al. Leukemia, 30(2): 484-491 (2016)).

The second binding domain of the trispecific antigen binding protein is designed to bind the cell surface immune checkpoint protein with low affinity to allow for rapid dissociation from the target. In this manner, the trispecific antigen binding protein may not engage with immune checkpoint proteins on healthy tissue, thereby avoiding off-target effects.

The binding affinity of the second binding domain of the trispecific antigen binding protein may be in the range of about 1 nM to about 100 nM. The binding affinity of the first binding domain may be in the range of about 1 nM to about 80 nM. The binding affinity of the first binding domain may be in the range of about 10 nM to about 80 nM.

Examples of bispecific antigen binding proteins with binding specificity to a cell surface immune checkpoint protein on a tumor cell includes, WO 2017106453 A1, WO 2017201281 A1, and Horn et al. Oncotarget, 8: 57964, 2017.

PD-L1 antigen binding domain sequences are recited below in Table 3 and in WO2017147383 and U.S. 20130122014 A1 as an example of binding domains capable of binding a cell surface immune checkpoint protein on a tumor cell. The sequences may be used in either a Fab or scFv format as part of the trispecific antigen binding protein.

TABLE 3
PD-L1 antigen binding domain sequences.

| SEQ | | |
|---|---|---|
| ID NO: | Sequence | Note |
| 35 | EVQLVESGGGLVQPGGSLRLSCTVSG | Anti-PD- |
| | IDLSSYTMGWVRQAPGKGLEWVGIIS | L1 VH |
| | SGGRTYYASWAKGRFTISRDTSKNTV | sequence |
| | YLQMNSLRAEDTAVYYCARGRYTGYP | |
| | YYFALWGQGTLVTVSS | |
| 36 | EIVMTQSPSTLSASVGDRVIITCQAS | Anti-PD- |
| | EDIYSLLAWYQQKPGKAPKLLIYDAS | L1 VL |
| | DLASGVPSRFSGSGSGAEFTLTISSL | sequence |
| | QPDDFATYYCQGNYGSSSSSSYGAVF | |
| | GQGTKLTVLG | |
| 37 | QVQLVQSGAEVKKPGSSVKVSCKTSG | Anti-PD-L1 |
| | DTFSTYAISWVRQAPGQGLEWMGGII | 12A4 VH |
| | PIFGKAHYAQKFQGRVTITADESTST | sequence |
| | AYMELSSLRSEDTAVYFCARKFHFVS | |
| | GSPFGMDVWGQGTTVTVSS | |
| 38 | EIVLTQSPATLSLSPGERATLSCRAS | Anti-PD-L1 |
| | QSVSSYLAWYQQKPGQAPRLLIYDAS | 12A4 VL |
| | NRATGIPARFSGSGSGTDFTLTISSL | sequence |
| | EPEDFAVYYCQQRSNWPTFGQGTKVE | |
| | IK | |

### Binding Domains to Cell Surface Proteins of Immune Cells

Trispecific antigen binding proteins having a third binding domain capable of binding to a cell surface protein of an immune cell are provided. The third binding domain of the trispecific antigen binding proteins are capable of recruiting immune cells specifically to the target tumor cells to be eliminated. Examples of immune cells that may be recruited include, but are not limited to, T cells, B cells, natural killer (NK) cells, natural killer T (NKT) cells, neutrophil cells, monocytes, and macrophages. Examples of surface proteins that may be used to recruit immune cells includes, but are limited to, CD3, TCRα, TCRβ, CD16, NKG2D, CD89, CD64, and CD32a. An exemplary cell surface protein of an immune cell is CD3.

Exemplary CD3 antigen binding domains are recited below in Table 4 and in WO2016086196 and WO2017201493, incorporated herein by reference.

TABLE 4
CD3 antigen binding domain sequences.

| SEQ | | |
|---|---|---|
| ID NO: | Sequence | Note |
| 39 | EVQLVESGGGLVQPGGSLRLSCAAS | Anti-CD3 VH |
| | GFTFSTYAMNWVRQAPGKGLEWVGR | sequence |
| | IRSKANNYATYYADSVKGRFTISRD | |
| | DSKNTLYLQMNSLRAEDTAVYYCVR | |
| | HGNFGDSYVSWFAYWGQGTLVTVSS | |
| 40 | AVVTQEPSLTVSPGGTVTLTCGSST | Anti-CD3 VL |
| | GAVTTSNYANWVQQKPGKSPRGLIG | sequence |
| | GTNKRAPGVPARFSGSLLGGKAALT | |
| | ISGAQPEDEADYYCALWYSNHWVFG | |
| | GGTKLTVL | |
| 41 | EVQLVESGGGLVQPGGSLKLSCAAS | Anti-CD3 VH |
| | GFTFNKYAINWVRQAPGKGLEWVAR | sequence |
| | IRSKYNNYATYYADQVKDRFTISRD | |
| | DSKNTAYLQMNNLKTEDTAVYYCVR | |
| | HANFGNSYISYWAYWGQGTLVTVSS | |
| 42 | QTVVTQEPSLTVSPGGTVTLTCASS | Anti-CD3 VL |
| | TGAVTSGNYPNWVQQKPGQAPRGLI | sequence |
| | GGTKFLVPGTPARFSGSLLGGKAAL | |
| | TLSGVQPEDEAEYYCTLWYSNRWVF | |
| | GGGTKLTVL | |
| 43 | QVQLQQSGAELARPGASVKMSCKAS | Anti-CD3 VH |
| | GYTFTRYTMHWVKQRPGQGLEWIGY | (OKT3) |
| | INPSRGYTNYNQKFKDKATLTTDKS | sequence |
| | SSTAYMQLSSLTSEDSAVYYCARYY | |
| | DDHYCLDYWGQGTTLTVSS | |
| 44 | QIVLTQSPAIMSASPGEKVTMTCSA | Anti-CD3 VL |
| | SSSVSYMNWYQQKSGTSPKRWIYDT | (OKT3) |
| | SKLASGVPAHFRGSGSGTSYSLTIS | sequence |
| | GMEAEDAATYYCQQWSSNPFTFGSG | |
| | TKLEIN | |

### Reduced Binding Affinity to the Cell Surface Immune Checkpoint Proteins of Tumor Cells and to Cell Surface Proteins of Tumor Cells

Trispecific antigen binding proteins have reduced binding affinity to the cell surface protein of a target tumor cell (e.g., BCMA) and reduced binding affinity to the cell surface immune checkpoint protein of the target tumor cell (e.g., PD-L1). The individual binding affinity of each binding domain is such that that the trispecific antigen binding protein may have reduced off-target binding to non-tumor or healthy tissue. On-target binding is improved when a target tumor cell expresses both the target immune checkpoint protein and the target cell surface protein. The combined binding avidity of the two domains is such that the trispecific antigen binding protein should bind the target tumor that expresses both antigens more specifically than healthy tissue. The trispecific antigen binding proteins do not have to rely on high affinity binding to the cell surface protein of a target tumor cell to achieve productive binding to the target tumor. By way of example, but in no way limiting, BCMA may be found on the surface of tumor cells and as a soluble form of the cell-surface antigen BCMA. BCMA is cleaved by y-secretase at the transmembrane region resulting in a soluble form of the BCMA extra-cellular domain (sBCMA). sBCMA may act as a decoy for the ligand APRIL and this serum soluble form of the cell-surface antigen BCMA may result in an antibody-antigen sink. High affinity anti-BCMA antibodies may therefore be more susceptible to sBCMA interference than a low affinity antibody (see, for example, Tai et al. Immunotherapy. 7(11): 1187-1199, 2015 and Sanchez et al. Br J Haematol. 158(6); 727738, 2012). By extension, other cell surface proteins on a target tumor cell may also be expressed on the surface of non-tumor cells. The presence of the cell surface proteins on non-tumor cells may act as an antibody-antigen sink, reducing the amount of antibody available to bind the tumor cells. Accordingly, therapeutic antibodies, such as the trispecific antigen binding proteins disclosed herein, may be less susceptible to the antibody-antigen sink if the antibodies possess low or medium binding affinity to the cell surface protein. This same principle may apply to the cell surface immune checkpoint protein of the target tumor cell as well.

### Expression of Antigen-Binding Polypeptides

In one aspect, polynucleotides encoding the binding polypeptides (e.g., antigen-binding proteins) disclosed herein are provided. Methods of making a binding polypeptide comprising expressing these polynucleotides are also provided.

Polynucleotides encoding the binding polypeptides disclosed herein are typically inserted in an expression vector for introduction into host cells that may be used to produce the desired quantity of the claimed antibodies, or fragments thereof. Accordingly, in certain aspects, the invention provides expression vectors comprising polynucleotides disclosed herein and host cells comprising these vectors and polynucleotides.

The term “vector” or “expression vector” is used herein to mean vectors used in accordance with the present invention as a vehicle for introducing into and expressing a desired gene in a cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (e.g., RSV, MMTV, MOMLV or the like), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the binding polypeptides may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese hamster ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney) and the like. In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). Host cell lines are typically available from commercial services, e.g., the American Tissue Culture Collection, or from published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the antigen binding proteins featured in the invention can also be expressed non-mammalian cells such as bacteria or yeast or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the proteins can become part of inclusion bodies. The proteins must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### Methods of Administering Antigen Binding Proteins

Methods of preparing and administering antigen binding proteins (e.g., trispecific antigen binding proteins disclosed herein) to a subject are well known to or are readily determined by those skilled in the art. The route of administration of the antigen binding proteins of the current disclosure may be oral, parenteral, by inhalation or topical. The term parenteral as used herein includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, the modified antibodies can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M or 0.05M phosphate buffer, or 0.8% saline. Other common parenteral vehicles include sodium phosphate solutions, Ringer's dextrose, dextrose and sodium chloride, lactated Ringer's, fixed oils and the like. Intravenous vehicles include, but are not limited to, fluid and nutrient replenishers, electrolyte replenishers, such as those based on Ringer's dextrose, and the like. Preservatives and other additives may also be present such as for example, antimicrobials, antioxidants, chelating agents, inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage, and should also be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. Isotonic agents, for example, sugars, polyalcohols, such as mannitol, sorbitol, or sodium chloride may also be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation typically include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture can include labels or package inserts indicating that the associated compositions are useful for treating a subject suffering from, or predisposed to autoimmune or neoplastic disorders.

Effective doses of the compositions of the present disclosure, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals, can also be treated. Treatment dosages may be titrated using routine methods known to those of skill in the art to optimize safety and efficacy.

For passive immunization with an antigen binding proteins, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example, dosages can be 1 mg/kg body weight or 10 mg/kg body weight or within the range of 1-10 mg/kg, e.g., at least 1 mg/kg. Doses intermediate in the above ranges are also intended to be within the scope of the current disclosure. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimens entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more antigen binding proteins with different binding specificities are administered simultaneously, in which case the dosage of each antigen binding protein administered falls within the ranges indicated.

Antigen binding proteins described herein can be administered on multiple occasions. Intervals between single dosages can be weekly, monthly or yearly. Intervals can also be irregular as indicated by measuring blood levels of modified binding polypeptide or antigen in the patient. In some methods, dosage is adjusted to achieve a plasma modified antigen binding protein concentration of 1-1000 ng/ml and in some methods 25-300 ng/ml. Alternatively, antigen binding protein can be administered as a sustained release formulation, in which case less frequent administration is required. For antigen binding proteins, dosage and frequency vary depending on the half-life of the antigen binding protein in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present antigen binding protein or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of antibody per dose, with dosages of from 5 to 25 mg being more commonly used for radioimmunoconjugates and higher doses for cytotoxin-drug modified antibodies) at relatively short intervals is sometimes required until progression of the disease is reduced or terminated, or until the patient shows partial or complete amelioration of disease symptoms. Thereafter, the patient can be administered a prophylactic regime.

Antigen binding proteins described herein can optionally be administered in combination with other agents that are effective in treating the disorder or condition in need of treatment (e.g., prophylactic or therapeutic). Effective single treatment dosages (i.e., therapeutically effective amounts) of ⁹⁰Y-labeled modified antibodies of the current disclosure range from between about 5 and about 75 mCi, such as between about 10 and about 40 mCi. Effective single treatment non-marrow ablative dosages of ¹³¹I-modified antibodies range from between about 5 and about 70 mCi, such as between about 5 and about 40 mCi. Effective single treatment ablative dosages (i.e., may require autologous bone marrow transplantation) of ¹³¹I-labeled antibodies range from between about 30 and about 600 mCi, such as between about 50 and less than about 500 mCi. In conjunction with a chimeric antibody, owing to the longer circulating half-life vis-a-vis murine antibodies, an effective single treatment of non-marrow ablative dosages of ¹³¹I labeled chimeric antibodies range from between about 5 and about 40 mCi, e.g., less than about 30 mCi. Imaging criteria for, e.g., an “In label, are typically less than about 5 mCi.

While the antigen binding proteins may be administered as described immediately above, it must be emphasized that in other embodiments antigen binding proteins may be administered to otherwise healthy patients as a first line therapy. In such embodiments the antigen binding proteins may be administered to patients having normal or average red marrow reserves and/or to patients that have not, and are not, undergoing one or more other therapies. As used herein, the administration of modified antibodies or fragments thereof in conjunction or combination with an adjunct therapy means the sequential, simultaneous, coextensive, concurrent, concomitant, or contemporaneous administration or application of the therapy and the disclosed antibodies. Those skilled in the art will appreciate that the administration or application of the various components of the combined therapeutic regimen may be timed to enhance the overall effectiveness of the treatment. A skilled artisan (e.g., an experienced oncologist) would be readily be able to discern effective combined therapeutic regimens without undue experimentation based on the selected adjunct therapy and the teachings of the instant specification.

As previously discussed, the antigen binding proteins of the present disclosure, immunoreactive fragments or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the modified antigen binding proteins, immunoreactive fragment or recombinant thereof, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the modified binding polypeptide will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

The biological activity of the pharmaceutical compositions defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of the invention, using e.g., standardized NCI response criteria. The success or in vivo efficacy of the therapy using a pharmaceutical composition of the invention refers to the effectiveness of the composition for its intended purpose, i.e., the ability of the composition to cause its desired effect, i.e., depletion of pathologic cells, e.g., tumor cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used. Furthermore, computer-aided tomography, X-ray, nuclear magnetic resonance tomography (e.g., for National Cancer Institute-criteria based response assessment [Cheson B D, Horning S J, Coiffier B, Shipp M A, Fisher R I, Connors J M, Lister T A, Vose J, Grillo-Lopez A, Hagenbeek A, Cabanillas F, Klippensten D, Hiddemann W, Castellino R, Harris N L, Armitage J O, Carter W, Hoppe R, Canellos G P. Report of an international workshop to standardize response criteria for non-Hodgkin's lymphomas. NCI Sponsored International Working Group. J Clin Oncol. 1999 April; 17(4):1244]), positron-emission tomography scanning, white blood cell counts, differentials, Fluorescence Activated Cell Sorting, bone marrow aspiration, lymph node biopsies/histologies, and various lymphoma specific clinical chemistry parameters (e.g., lactate dehydrogenase) and other established standard methods may be used.

### Methods of Treating Cancer

Methods of treating cancer using the trispecific antigen binding proteins described herein in a subject suffering from cancer are provided. Methods of targeting and killing tumor cells using the trispecific antigen binding proteins described herein are also provided.

The first binding domain of the trispecific antigen binding protein of the invention specifically binds to a cell surface protein that is associated to the tumor cell. In an exemplary embodiment, the cell surface tumor protein is absent or significantly less abundant in healthy cells relative to the tumor cells. The trispecific antigen binding protein of the invention preferentially attaches to the tumor cells carrying such tumor antigens. Examples of cell surface proteins associated to certain tumor cells include, but are not limited to, CD33 (a cell surface protein that is highly expressed on AML (acute myeloid leukemia) cells), CD20 (a cell surface protein expressed on B cell lymphomas and leukemias), BCMA (a cell surface protein expressed on multiple myeloma cells), CD19 (a cell surface protein expressed on ALL (acute lymphoblastic leukemia)), and the like.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1

### Design, Expression and Purification of Exemplary Trispecific Antigen Binding Proteins

### Background

A major challenge in developing trispecific antigen binding protein therapeutics is the selection of a molecular format from structurally diverse alternatives that can support a wide range of different biologic and pharmacologic properties while maintaining desirable attributes for developability. Such attributes include high thermal stability, high solubility, low propensity to aggregate, low viscosity, chemical stability and high-level expression (grams per liter titers).

Production of trispecific antigen binding proteins by co-expression of multiple (three) light and heavy chains in a single host cell can be highly challenging because of the low yield of the desired trispecific antigen binding protein and the difficulty in removing closely related mispaired contaminants. In IgG-based trispecific antigen binding proteins, the heavy chains form homodimers as well as the desired heterodimers. Additionally, light chains can mispair with non-cognate heavy chains. Consequently, co-expression of multiple chains can result in many unwanted species (other than the desired trispecific antigen binding protein) and therefore low production yields.

### Selection of Antibodies for Construction of Trispecific Molecules

Two different anti-CD3 antibodies derived from SP34 and OKT3 were used as binding arms to CD3 for the construction of bispecific and trispecific molecules. Both antibodies are characterized by their ability of activating T-cells and have been used in the generation of therapeutic bispecific antibodies that can be used in the treatment of cancer.

For the neutralization of the PD-1/PD-L1 pathway, a major mechanism of tumor immune evasion, the anti-PD-L1 antibodies KN035 (Cell Discov. 2017; 3: 17004) and SEQ ID NO: 9 of the patent application WO2017147383 were chosen for the generation of bispecific and trispecific antibodies. The mouse antibody C 11D5.3 and a single domain antibody, 269A37346 (described in WO2018028647) were used as entities targeting BCMA in the construction of the bispecific and trispecific molecules. C11D5.3 binds specifically to BCMA on the surface of one or more subset of B cells including plasma cells as well as the soluble receptor and, also efficiently binds BCMA expressed on multiple myeloma and plasmacytomas (described in WO2016094304A2). Additional antibodies against Tumor-Associated Antigens (TAA) include Trastuzumab, an anti-HER2 humanized monoclonal antibody for the treatment of HER2-positive metastatic breast cancer (Cho et al. Nature, 421(6924): 756-760 (2003)) and Blinatumomab, a bispecific T-cell engager monoclonal antibody indicated for the treatment of Philadelphia chromosome-negative relapsed or refractory B-cell precursor acute lymphoblastic leukemia (ALL).

### Assembly of Trispecific Molecules and Bispecific Controls

The anti-BCMA antibody Cl 1D5.3, the anti-PD-L1 antibody of SEQ ID NO: 9 of the patent application WO2017147383 and the anti-CD3 antibody SP34 were chosen for construction of bispecific and trispecific antibodies which were assembled in two different formats: 1) a tandem scFv fusion which comprises two scFv fragments connected by a peptide linker on a single protein chain; and 2) scFv fusions to the C-terminal chains of a Fab where the scFvs were assembled as either light or heavy chain C-terminal fusions of the Fab portion. The Fab format, which is highly stable and an efficient heterodimerization scaffold, was used to produce recombinant bispecific and trispecific antibody derivatives (Schoonjans et al. J Immunol. 2000 Dec. 15; 165(12):7050-7). Table 5 below lists the constructs and positions of binding moieties as either tandem scFv fusions or scFvs linked to the C-terminal of Fab molecules.

TABLE 5
Antibody formats.

| | | SEQ ID |
|---|---|---|
| ID | Description | NO |
| CDR1-005 | CD3-binding scFv linked to C-terminal light | 9, 10 |
| | chain of BCMA-binding Fab | |
| CDR1-006 | anti-CD3 scFv linked to the C-terminal light | 10, 11 |
| | chain and anti-BCMA scFv linked to the C terminal | |
| | heavy chain of an anti-BCMA Fab | |
| CDR1-007 | anti-CD3 scFv linked to the C-terminal light chain | 10, 12 |
| | and anti-PD-L1 scFv linked to the C terminal heavy | |
| | chain of an anti-BCMA Fab | |
| CDR1-008 | CD3-binding scFv linked to the C-terminal of | 13 |
| | BCMA-binding scFv | |
| CDR1-020 | CD3-binding scFv linked to C-terminal light | 12, 14 |
| | chain of inactive Fab and PD-L1-binding scFv | |
| | linked to C-terminal heavy chain of inactive Fab | |
| CDR1-061 | anti-CD3 scFv linked to the C-terminal light | 27, 28 |
| | chain and anti-PD-L1 scFv linked to the C | |
| | terminal heavy chain of an anti-CD19 Fab | |
| CDR1-063 | CD3-binding scFv linked to the C-terminal | 63 |
| | of CD19-binding scFv | |
| CDR1-081 | anti-CD3 scFv linked to the C-terminal | 30, 31 |
| | light chain and anti-PD-L1 scFv linked | |
| | to the C terminal heavy chain of an anti-HER2 Fab | |
| CDR1-083 | CD3-binding scFv linked to C-terminal light | 30, 32 |
| | chain of HER2-binding Fab | |

### Position of the Antigen Binding Sites in Fab-scFv Trispecific Molecules

To investigate whether the position of the antigen binding sites could affect the binding activity and/or efficiency to redirect immune cell killing to a tumor cell, Fab-scFv fusions were constructed to explore each antigen binding site in 3 possible positions: 1) Fab; 2) scFv linked to the C terminal of the Fab light chain; and 3) scFv linked to the C terminal of the Fab heavy chain. Table 6 below lists the constructs with binding moieties in different positions.

TABLE 6
Antibody formats.

| | | SEQ ID |
|---|---|---|
| ID | Description | NO |
| CDR1-007 | anti-CD3 scFv linked to the C-terminal light | 10, 12 |
| | chain and anti-PD-L1 scFv linked to the C terminal | |
| | heavy chain of an anti-BCMA Fab | |
| CDR1-047 | anti-PD-L1 scFv linked to the C-terminal light | 15, 16 |
| | chain and anti-CD3 scFv linked to the C terminal | |
| | heavy chain of an anti-BCMA Fab | |
| CDR1-048 | anti-PD-L1 scFv linked to the C-terminal light chain | 17, 18 |
| | and anti-BCMA scFv linked to the C terminal heavy | |
| | chain of an anti-CD3 Fab | |
| CDR1-049 | anti-BCMA scFv linked to the C-terminal light | 19, 20 |
| | chain and anti-CD3 scFv linked to the C terminal | |
| | heavy chain of an anti-PD-L1 Fab | |

### Design of Alternative Trispecific Formats

To investigate whether other antibody formats or different antigen binding sequences could fulfill the requirements for generating the trispecific antibodies of the invention (e.g., matching valency with biology, retention of the binding activity to different targets, the ability to bind different targets simultaneously and to physically link an immune cell to a tumor cell), exemplary trispecific molecules were assembled using different binding sequences, different formats (e.g., scFvs, sdAbs, Fabs or Fc-based) and combinations thereof.

For Fab based constructs, scFvs or sdAbs were fused to the C-terminal regions of the Fab. For Fc based constructs, the scFvs were assembled as either N- or C-terminal fusions to the Fc region or to the C-terminal region of the light chain. The knobs-into-holes (KIHs) technology was used to promote heterodimerization of the Fc portions and avoid mispairing of the chains which would prevent the right formation of the trispecific molecules. Table 7 below lists the constructs with alternative trispecific formats.

TABLE 7
Antibody formats.

| | | SEQ ID |
|---|---|---|
| ID | Description | NO |
| CDR1-055 | anti-CD3 scFv linked to the C-terminal light | 21, 10 |
| | chain and anti-PD-L1 sdAb linked to the C | |
| | terminal heavy chain of an anti-BCMA Fab | |
| CDR1-056 | a tandem scFv-sdAb-sdAb fusion: N-terminal | 22 |
| | CD3-binding scFv linked to BCMA-binding sdAb | |
| | linked to PD-L1 sdAb C-terminal | |
| CDR1-057 | anti-CD3 scFv linked to the C-terminal light chain | 10, 23, 24 |
| | of the anti-BCMA Fd portion of the “knob” | |
| | arm and an anti-PD-L1 scFv linked to the | |
| | N-terminal of the “hole arm” | |
| CDR1-058 | anti-CD3 scFv linked to the C-terminal of the | 24, 25, 26 |
| | anti-BCMA “knob” arm and an anti-PD-L1 | |
| | scFv linked to the N-terminal of the “hole | |
| | arm” | |
| CDR1-081 | anti-CD3 OKT3 scFv linked to the C-terminal | 12, 43, 44 |
| | light chain and anti-PD-L1 scFv linked to the | |
| | C terminal heavy chain of an anti-BCMA Fab | |

### Expression

Synthetic genes encoding for the different antibody chains (i.e., heavy chain and light chain) were constructed at Twist Bioscience Corporation and were separately cloned into the expression vectors for transient expression in HEK 293 6E cells. Expression vector DNA was prepared using conventional plasmid DNA purification methods (for example Qiagen HiSpeed plasmid maxi kit, cat. # 12662). Several exemplary trispecific antigen binding protein formats expressed in HEK293-6E cells to evaluate yield and purity of each specific format.

The trispecific antigen binding proteins and bispecific antigen binding protein controls were expressed by transient co-transfection of the respective mammalian expression vectors in HEK293-6E cells, which were cultured in suspension using polyethylenimine (PEI 40 kD linear). The HEK293-6E cells were seeded at 1.7×10⁶ cells/mL in Freestyle F17 medium supplemented with 2 mM L-Glutamine. The DNA for every mL of the final production volume was prepared by adding DNA and PEI separately to 50 μL medium without supplement. Both fractions were mixed, vortexed and rested for 15 minutes, resulting in a DNA : PEI ratio of 1 : 2.5 (1 μg DNA/mL cells). The cells and DNA/PEI mixture were put together and then transferred into an appropriate container which was placed in a shaking device (37° C., 5% CO₂, 80% RH). After 24 hours, 25 μL of Tryptone N1 was added for every mL of final production volume.

After 7 days, cells were harvested by centrifugation and sterile filtrated. The antigen binding proteins were purified by an affinity step. For the affinity purification of Fab-based constructs, the supernatant was loaded on a protein CH column (Thermo Fisher Scientific, #494320005) equilibrated with 6 CV PBS (pH 7.4). Tandem scFvs were purified using a Capto L column, GE Healthcare, # 17547815. After a washing step with the same buffer, the antigen binding protein was eluted from the column by step elution with 100 mM Citric acid (pH 3.0). The fractions with the desired antigen binding protein were immediately neutralized by 1 M Tris Buffer (pH 9.0) at 1:10 ratio, then pooled, dialyzed and concentrated by centrifugation.

After concentration and dialysis against PBS buffer, content and purity of the purified proteins were assessed by SDS-PAGE and size-exclusion HPLC. After expression in HEK293-6E cells, the proteins were purified by a single capture step and analyzed by analytical size exclusion chromatography.

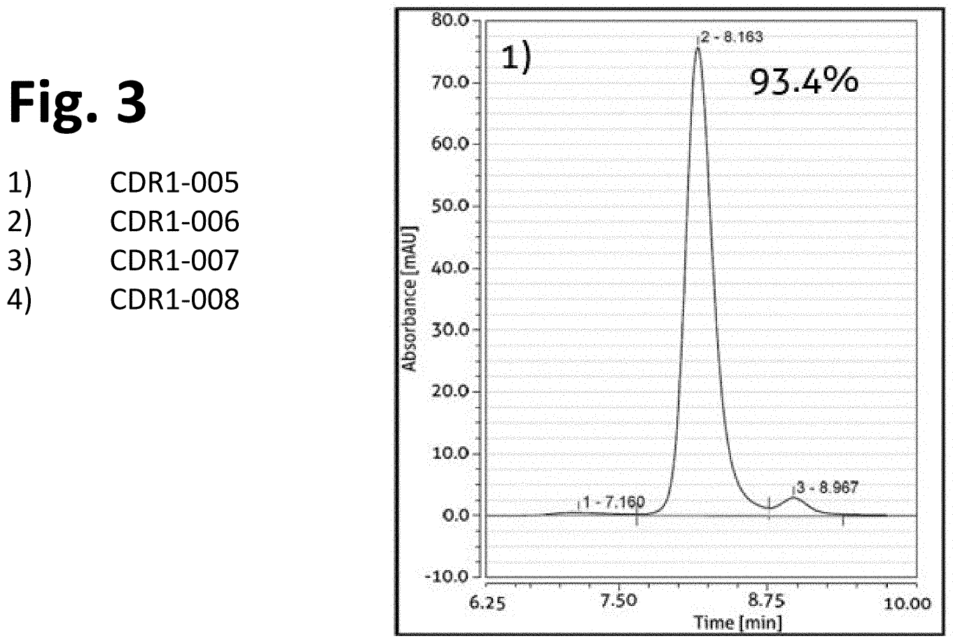

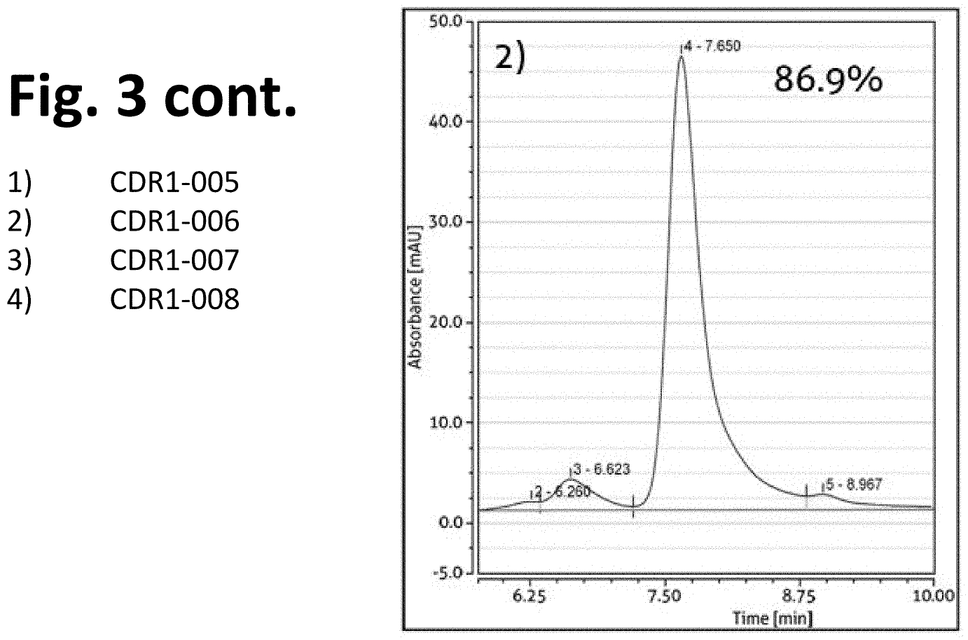

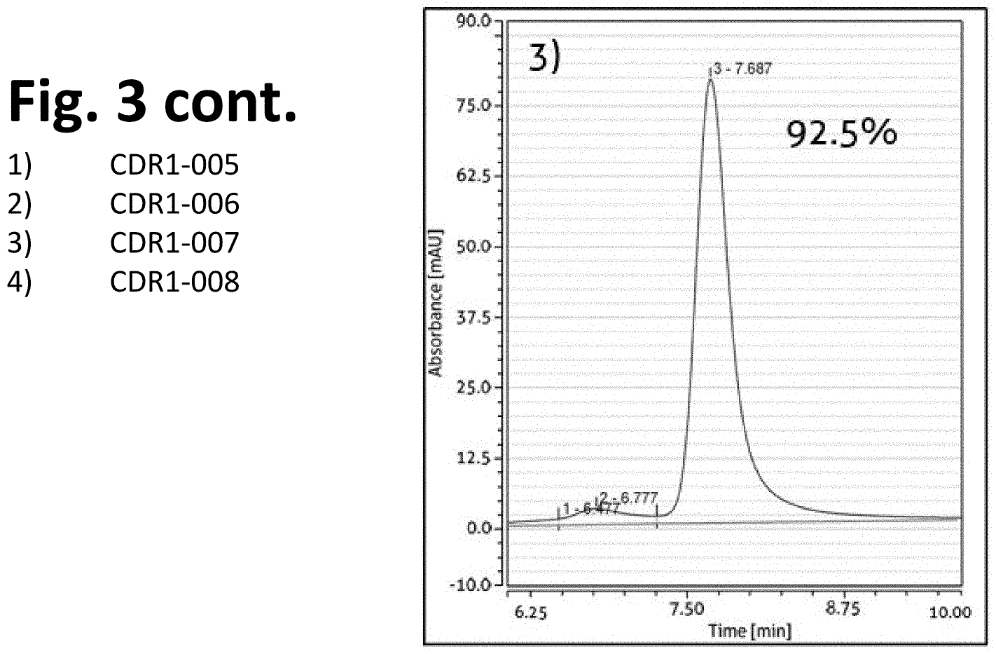

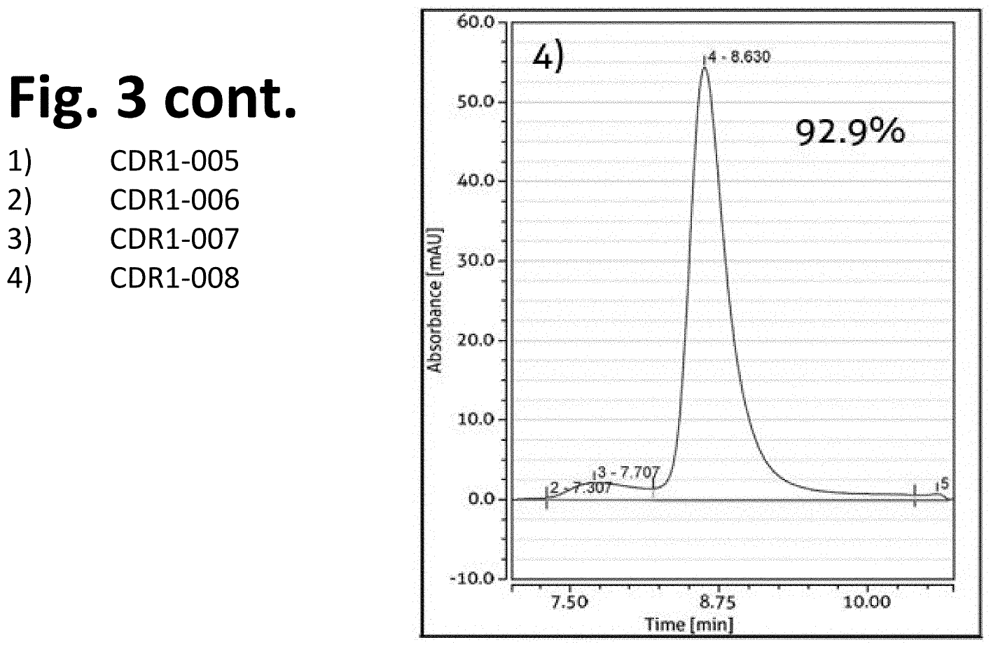

FIG. 2 depicts a variety of multi-functional proteins that feature one or several scFv and/or Fab modules attached together in different combinations. scFv fragments exhibited great variability in their stability, expression levels and aggregation propensity. Accordingly, molecules 001-004 were used as a reference as they are derived from scFvs fragments with favorable biophysical properties (J Biol Chem. 2010 Mar. 19; 285(12): 9054-9066). The results showed that the various bispecific and trispecific formats were expressed at high levels in mammalian cells, the antigen binding proteins were mostly in monomeric form, and there was no observable clipping or fragmentation of the proteins (FIG. 3).

### Example 2

### Ability of the Trispecific Molecules and Bispecific Controls to Bind Their Targets

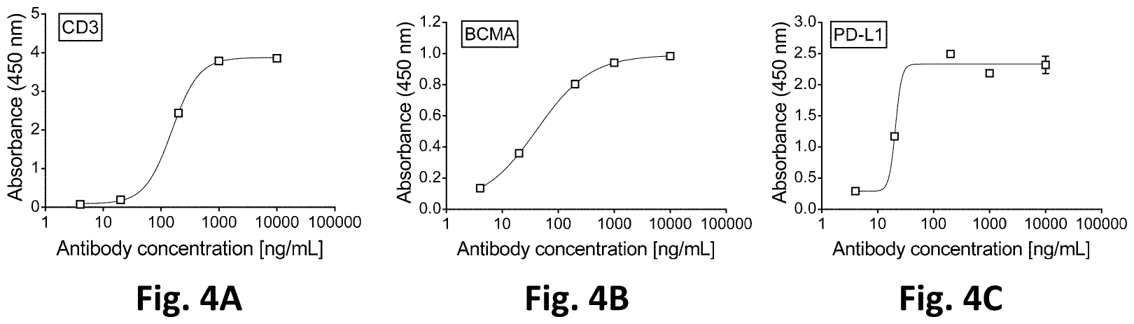

Binding ELISA assays were performed to determine if the exemplary trispecific antigen binding proteins bound to their respective targets. The trispecific antibody CDR1-007 was evaluated for its ability to bind its antigens. Serial dilutions of CDR-007 to final concentrations ranging from 4 ng/mL to 10 μg/ml were tested in ELISA for binding to the extracellular domain of human PD-L1 His-tag (Novoprotein, #C315), recombinant Human BCMA Fc Chimera (produced in-house via transient expression in HEK293-6E cells) and CD3 epsilon His-tag (Novoprotein, #C578), each of which was coated on a 96 well plate. The trispecific antibody was detected by goat anti-kappa-LC antibody HRP (Thermo Fisher Scientific, #A18853). FIG. 4A-4C shows concentration-dependent binding of CDR1-007, confirming the ability of the trispecific antibody to bind the three targets.

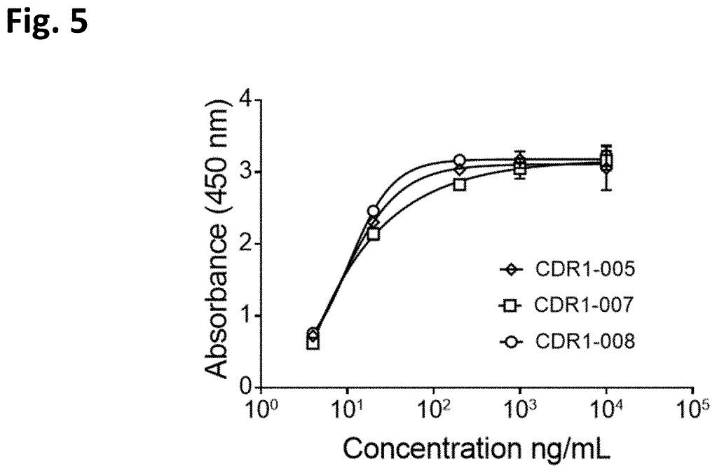

In addition, trispecific and bispecific antibodies were assessed for their ability to bind BCMA and CD3 simultaneously using a Dual-Binding ELISA. Briefly, serial dilutions of the antibody molecules CDR1-005, CDR1-007 and CDR1-008 were added to 96 well ELISA plates coated with recombinant human BCMA Fc Chimera (expressed after transient transfection in HEK293-6E) and followed by a secondary association with recombinant human CD3 epsilon His-tag protein (Novoprotein, Cat. No. C578). Simultaneous binding to antigen pairs was detected using an anti-His antibody (Abcam, Cat. No. ab1187). FIG. 5 shows concentration-dependent binding to BCMA and CD3 of bispecific and trispecific molecules. These data confirmed the bispecific and trispecific antibodies bound BCMA and CD3 simultaneously in a comparable manner.

### Example 3

### Ability of the CD3-Binding Arm to Induce Proliferation of T Cells

The antigen receptor molecules on human T lymphocytes were noncovalently associated on the cell surface with the CD3 (T3) molecular complex. Perturbation of this complex with anti-CD3 monoclonal antibodies could induce T cell activation, but this ability is dependent on certain properties such as binding affinity, epitope, valency, antibody format, etc.

Linking different antigen binding sites in fusion proteins to produce bispecific antibodies often exhibit reduced affinity for their target antigens compared to the parental antibodies. Therefore, careful consideration should be given during assessment of the CD3-binding arm of T cell engagers to ensure functionality. One of the most common ways to assess the ability of CD3 agonistic antibodies to activate T cells is to measure T cell proliferation upon in vitro stimulation.

The CD3-binding arm design of the invention was analyzed for its ability to trigger cell proliferation of CD3+ Jurkat T cells. The antibody CDR1-005 was coated on a 96-well plate surface to final concentrations ranging from 0.01 to 1 μg/mL. Anti-CD3 immobilized on a plate surface facilitated crosslinking of CD3 on T cells and thus was a better stimulant than soluble antibody. Jurkat T cell leukemic line E6-1 cells were adjusted to 1×10⁶ (viable) cells per ml in complete RPMI medium, 100 μl of this cell suspension was pipetted into a 96-well plate with immobilized anti-CD3 with and without antibody as a negative control and incubated at 37° C. and 5% CO₂ for 48 hours. After this incubation period, 10 μl per well of WST-1 cell proliferation reagent (Roche, Cat. No. 5015944001) was added to the cultures and incubated at 37° C. and 5% CO₂ for up to 5 hours. The formazan dye formed was measured at several timepoints up to 5 hours incubation at 450 nm and 620 nm as reference wavelength.

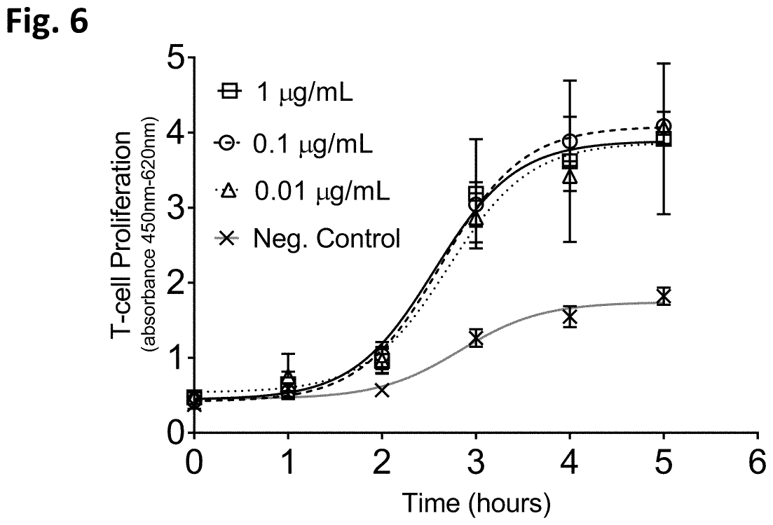

As depicted in FIG. 6, the formazan dye formation reached its maximum after 5 hours incubation and indicated that Jurkat T cells stimulated with the CD3-binding arm in CDR1-005 proliferated more than those without anti-CD3 stimulation, even at the lowest concentration of 0.1 μg/mL. This confirmed the suitability of the CD3-binding arm design to induce T cell activation.

### Example 4

### Trispecific Antibody Mediated IL-2 Cytokine Production of Jurkat T Cells in the Presence or Absence of Human Multiple Myeloma Cells

The trispecific antibody CDR1-007 was analyzed for its ability to induce IL-2 cytokine production in Jurkat T-cells upon engagement of myeloma cancer cells. Jurkat E6-1 T cells (effector) were co-incubated with NCI-H929 human multiple myeloma cells (target) or human embryonic kidney (HEK) 293 cells in the presence of 10, 100 or 200 nM CDR1-007, with an effector to target cell ratio of 5:1. Additionally, Jurkat E6-1 T cells were co-incubated with and without 1 ug/mL phytohemagglutinin (PHA) for unspecific stimulation of T cells as positive control.

After incubation for 18 hours at 37° C., 5% CO₂, the assay plate was centrifuged for 10 minutes at 1000×g and the supernatant was transferred onto a new 96-well plate for the subsequent analysis. The quantification of human IL-2 cytokine was performed using the Human IL-2 ELISA Kit (Thermo Fisher Scientific, Cat. No. 88-7025) according to the manufacturer's instructions.

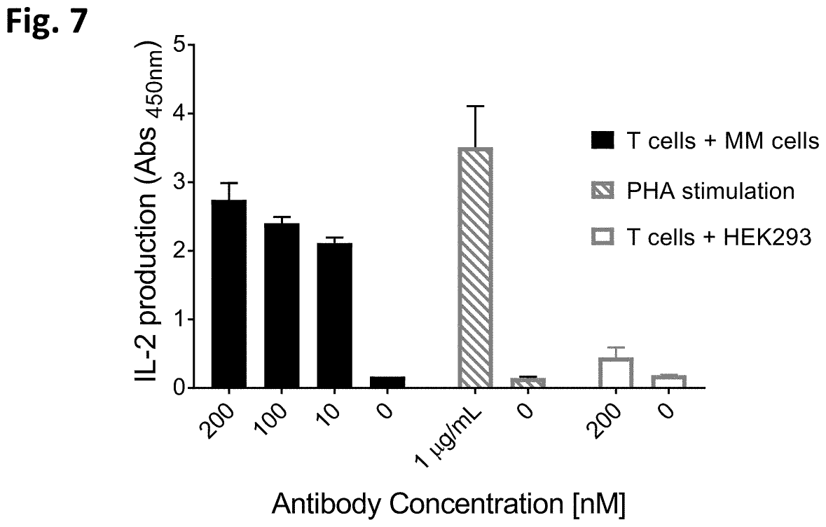

As shown in FIG. 7, the trispecific antibody CDR1-007 potently induced IL-2 cytokine production by Jurkat T cells upon engagement of H929 myeloma cells. CDR1-007 did not induce IL-2 production by Jurkat T cells when co-incubated with HEK293 cells, demonstrating that the activity of CDR1-007 was triggered upon engagement of cancer cells.

### Example 5

### Ability of Trispecific and Bispecific Antibodies to Induce IL-2 Cytokine Production Upon Binding to Human CD3+ T Cells and H929 Multiple Myeloma Cells

The trispecific antibody CDR1-007 was compared head-to-head to a bispecific tandem scFv BCMA-CD3 (CDR1-008) for the ability to induce IL-2 cytokine production in isolated human CD3+ T-cells upon engagement of myeloma cancer cells. Briefly, human CD3+ T cells were isolated from PBMCs using EasySep Human T Cell Isolation Kit (Stemcell, Cat. No. 17911) according to the manufacturer's instructions. 1×10⁵ isolated CD3+ T cells (effector) were co-incubated with NCI-H929 human multiple myeloma cells (target) at effector to target cell ratio of 5:1, in the presence of antibody with concentrations ranging from 1 to 100 nM. After incubation for 18 hours at 37° C., 5% CO₂, the assay plate was processed as described in Example 4 above.

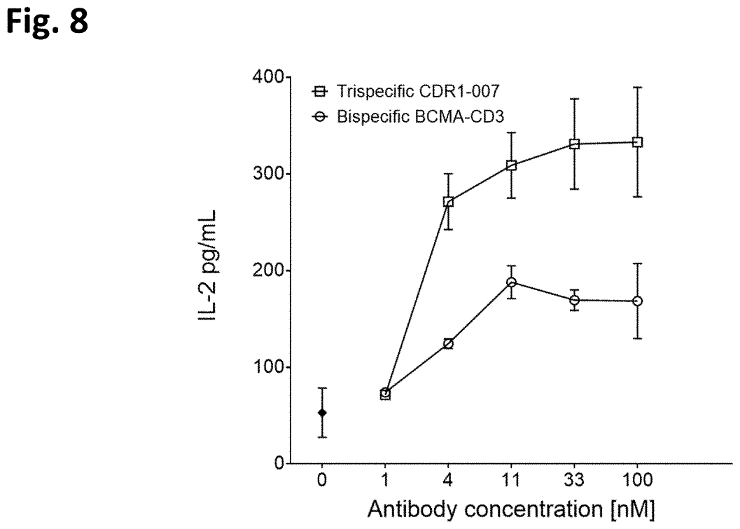

As depicted in FIG. 8, the trispecific antibody CDR1-007 induced concentration-dependent production of IL-2 cytokine by the isolated human T cells more efficiently than the bispecific CDR1-008. These results indicated that the additional binding site for PD-L1 in the trispecific antibody CDR1-007 contributed to a more potent T cell activation compared with the bispecific CDR1-008.

### Example 6

### Antibody Mediated Redirected T-Cell Cytotoxicity of H929 Myeloma Cells (LDH Release Assay)

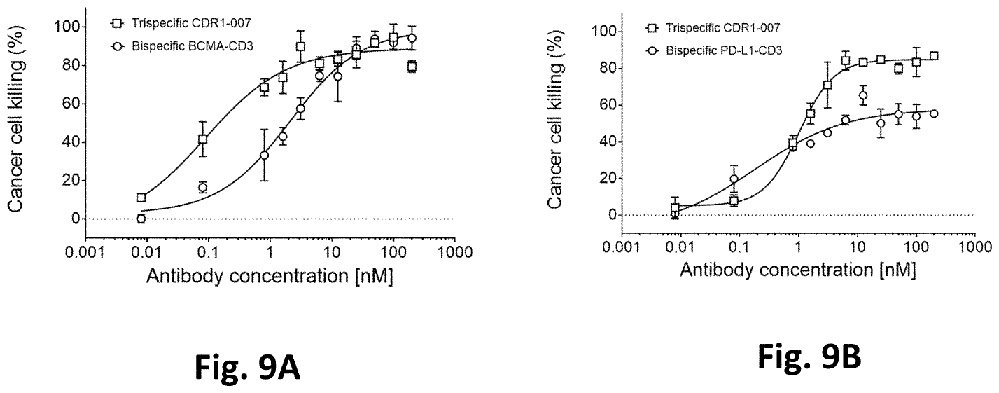

The trispecific antibody CDR1-007 was compared head-to-head with bispecific antibodies BCMA/CD3 (CDR1-008—FIG. 9A) and PD-L1/CD3 (CDR1-020—FIG. 9B) for the ability to induce T cell-mediated apoptosis of H929 human multiple myeloma cells. Briefly, isolated human CD3+ T cells and NCI-H929 human multiple myeloma cells were co-incubated as described in Example 5 in the presence of either the bispecific or trispecific antibody. For accurate comparison, all antibody constructs were adjusted to the same molarity in final concentrations ranging from 8 pM to 200 nM.

After 24 hours incubation at 37° C., 5% CO₂, T cell-mediated cytotoxicity of human myeloma cells was measured using the Pierce LDH Cytotoxicity Assay Kit (Thermo Fisher Scientific, Cat. No. 88954). For normalization, maximal killing of H929 human multiple myeloma cells (corresponding to 100% release of LDH) was obtained by incubating the same number of H929 cells used in experimental wells (20,000 cells) with lysis buffer. Minimal lysis was defined as LDH released by H929 cells co-incubated with CD3+ T cells without any test antibody. Concentration-response curves of H929 myeloma cell killing mediated by the antibodies were obtained by plotting the normalized LDH release values against the concentrations of trispecific and bispecific antibodies. The EC50 values were calculated by fitting the curves to a 4-parameter non-linear regression sigmoidal model with Prism GraphPad software.

As depicted in FIG. 9A and 9B, the trispecific antibody CDR1-007 induced more potently lysis of H929 myeloma cells than its bispecific counterparts CDR1-008 and CDR1-020. These results suggest a synergistic effect of targeting BCMA combined with PD-L1 blockade which results in more potent and effective T cell-mediated killing of cancer cells compared to the bispecific constructs targeting only cancer cell antigen.

### Example 7

### Other Trispecific Molecules

Whether the effects of the trispecific CDR1-007 described in the previous examples are transferable to: 1) trispecific Fab-scFv where each binding site is evaluated at different positions; and 2) alternative antibody formats and/or different antigen binding sequences, was next investigated.

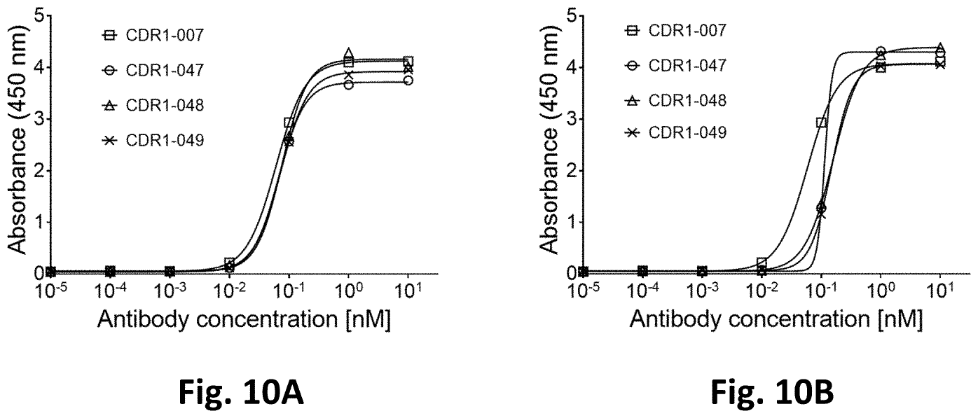

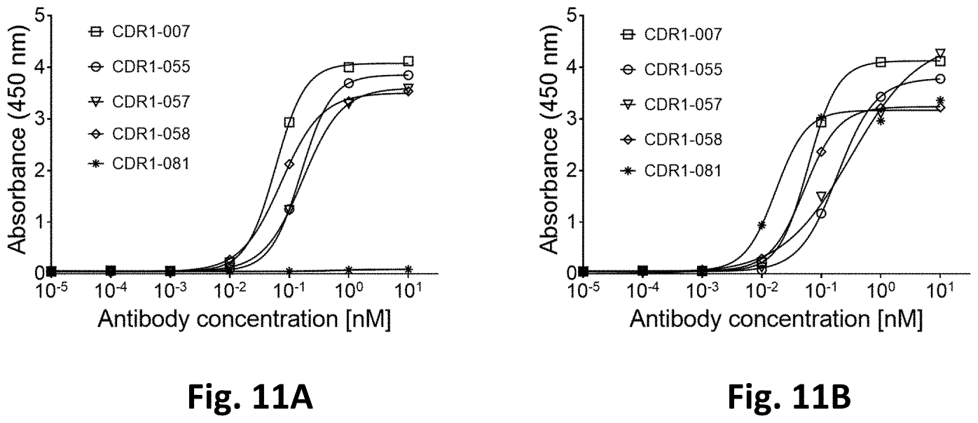

Trispecific antibody molecules were tested for their ability to bind the different targets using a Dual-Binding ELISA. Briefly, serial dilutions of the trispecific molecules (and the CDR1-007 control) to final concentrations ranging from 0.01 pM to 10 nM were added to 96 well ELISA plates coated with recombinant human BCMA Fc Chimera (expressed after transient transfection in HEK293-6E) and followed by a secondary association with either recombinant human CD3 epsilon His-tag protein (Novoprotein, Cat. No. C578) or recombinant human PD-L1 His-tag protein (expressed after transient transfection in HEK293-6E). Simultaneous binding to antigen pairs was detected via an anti-His antibody (Abcam, Cat. No. ab1187). FIGS. 10A and 10B showed concentration-dependent binding to BCMA-PD-L1 (FIG. 10A) and BCMA-CD3 (FIG. 10B) of trispecific molecules where the position of each binding site was evaluated in Fab-scFv constructs. FIGS. 11A and 11B showed concentration-dependent binding to BCMA-PD-L1 (FIG. 11A) and BCMA-CD3 (FIG. 11B), which evaluated alternative antibody formats and different antigen binding sequences. These data confirmed the ability of the trispecific antibodies to retain binding activity to the three different targets.

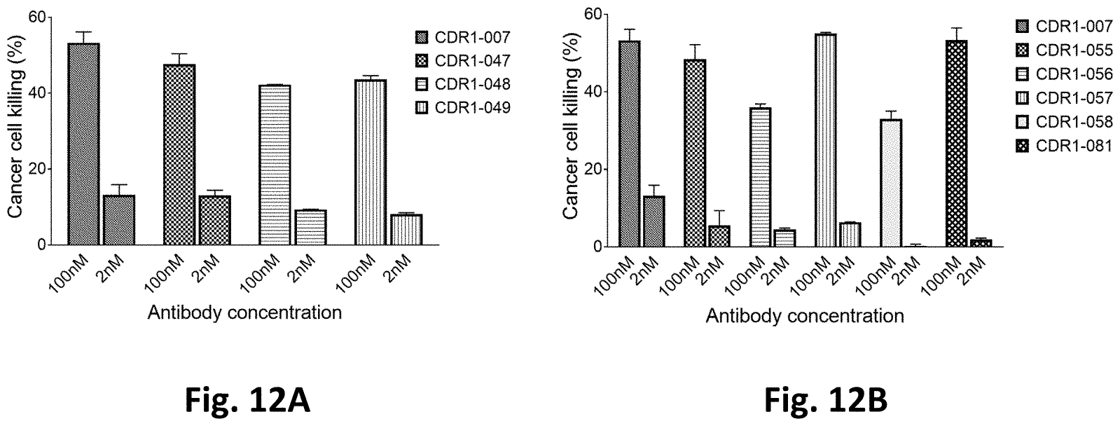

Next, the different trispecific constructs were evaluated for the ability to induce T cell-mediated killing of H929 human multiple myeloma cells. Trispecific antibodies at final concentrations of 100 nM and 2 nM were incubated with isolated human CD3+ T cells and NCI-H929 human multiple myeloma cells as described in Example 6. Most alternative trispecific molecules were found capable of inducing T-cell-mediated killing of H929 multiple myeloma cells in a comparable manner (FIGS. 12A and 12B).

### Example 8

### Anti-PD-L1 Antibody Affinity Variants

The above described examples showed that blockade of PD-L1 signal could synergize with the anti-BCMA (tumor antigen-binding arm) and the CD3-binding arm of trispecific antibodies to potently eliminate tumors. While PD-L1 was overexpressed on cancer cells, its expression in many normal tissues might result in on-target, off-tumor toxicities or create an antigen sink that could minimize the therapeutic efficacy of the trispecific antibodies. In this example, trispecific T cell engager antibodies that co-targeted PD-L1 and BCMA on cancer cells with reduced affinity for PD-L1 were generated. These characteristics facilitated selective binding of trispecific antibodies to tumor cells.

Briefly, a molecular model for the PD-L1 binding arm of CDR1-007 was generated using a fully automated protein structure homology-modeling server (website: expasy.org/swissmod), solvent exposed residues at CDR regions deemed to be important for binding were selected for mutation to alanine (M.-P.Lefranc, 2002; website: imgt.cines.fr, A. Honegger, 2001; website: unizh.ch/antibody). Table 8 shows the alanine mutations introduced at the CDR-regions of CDR1-007 as candidates to reduce the affinity of the PD-L1 binding-arm. Alanine mutations were generated using ten nanograms of CDR1-007 expression vectors as template, 1.5 μl mutated primers at 10 μmol and the Q5 Site-Directed Mutagenesis Kit (New England Biolabs, Cat. No. E0554S), used according to manufacturer's instructions. The resultant mutants were co-transfected in HEK293-6E cells and cultured for expression of the trispecific mutants as described in example 1. Serial dilutions of the antibodies to final concentrations ranging from 0.5 ng/mL to 50 μg/ml were tested by ELISA for binding to the extracellular domain of human PD-L1 coated on a 96 well plate.

TABLE 8
Alanine mutations introduced at CDR
regions of the PD-L1 binding arm.
Alanine mutations are shown in bold
underlined text.

| ID | CDR-L1 | CDR-L2 | CDR-L3 | CDR-H1 | CDR-H2 | CDR-H3 |
|---|---|---|---|---|---|---|
| CDR1- | QASEDI | DASDLA | QGNYGS | IDLSSY | IISSGG | GRYTGY |
| 007 | YSLLA | S | SSSSSY | TMG | RTYYAS | PYYFAL |
| | | | GAV | | WAKG | |
| CDR1- | QASEAI | | | | | |
| 010 | YSLLA | | | | | |
| CDR1- | QASEDI | | | | | |
| 011 | ASLLA | | | | | |
| CDR1- | | AASDLA | | | | |
| 012 | | S | | | | |
| CDR1- | | | QGAYGS | | | |
| 013 | | | SSSSSY | | | |
| | | | GAV | | | |
| CDR1- | | | | IDLSSY | | |
| 014 | | | | AMG | | |
| CDR1- | | | | | IISSAG | |
| 015 | | | | | RTYYAS | |
| | | | | | WAKG | |
| CDR1- | | | | | IISSGG | |
| 016 | | | | | ATYYAS | |
| | | | | | WAKG | |
| CDR1- | | | | | | GAYTGY |
| 017 | | | | | | PYYFAL |
| CDR1- | | | | | | GRATGY |
| 018 | | | | | | PYYFAL |

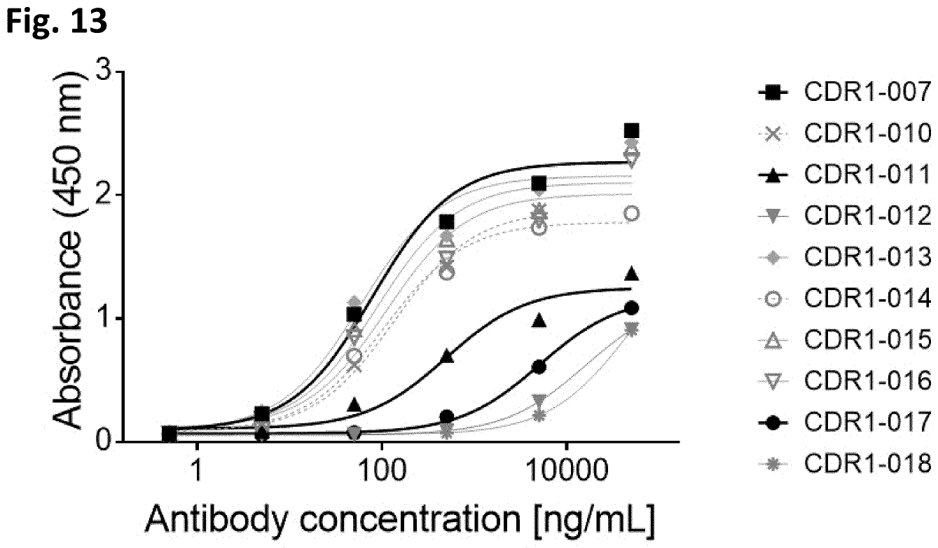

As depicted in FIG. 13, the concentration-response curves of the trispecific mutants showed different binding profiles to immobilized PD-L1, indicating a broad range of binding affinities. Trispecific molecules CDR1-007, CDR1-011 and CDR1-017 were considered to represent high, mid, and low affinity ranges and were selected for affinity characterization in solution by competition ELISA as described by Friguet et al. (J Immunol Methods. 1985 Mar. 18; 77(2):305-19). First, mixtures of the trispecific antibody (Ab) at a fixed concentration and the PD-L1 antigen (Ag) at varying concentrations were incubated for sufficient time to reach equilibrium. Then the concentration of trispecific antibody, which remained unsaturated at equilibrium (not associated with PD-L1 antigen), was measured by a classical indirect ELISA using PD-L1 coated plates. The amount of antigen coated in the wells and the incubation time for the ELISA were such that during the ELISA, equilibrium in solution was not significantly modified to avoid dissociation of trispecific-PD-L1 complex (X). The K_{d} was calculated from a Scatchard plot using the following equation:

[x]/[Ag]=([Ab]−[x])/Kd

TABLE 9
K_{d} values for select trispecific antibodies

| | | | | | Antigen |
|---|---|---|---|---|---|
| | | | R² | Antibody | concentration |
| | | | Scatchard | concentration | range |
| Molecule | K_{d} | | plot | (Ab) | (Ag) |
| CDR1-007 | 110 | pM | 1.00 | 5.0E−9M | 5.0E−8 to |
| | | | | | 4.9E−11M |
| CDR1-011 | 5 | nM | 0.82 | 1.0E−10M | 5.0E−7 to |
| | | | | | 4.9E−10M |
| CDR1-017 | 26 | nM | 0.99 | 5.0E−10M | 1.0E−6 to |
| | | | | | 9.8E−10M |

(Ab): trispecific antibody at a fixed concentration;
(Ag): PD-L1 antigen concentration range

To confirm the affinity measurements, the binding affinity of the anti-PD-L1 binding-arms of trispecific constructs CDRI -007 and CDRI -017 was also determined by Kinetic Exclusion Assay (KinExA®) using a KinExA 3200 (Sapidyne Instruments, USA) flow fluorimeter. Studies were designed to measure the free antibody in samples with a fixed antibody concentration and different concentrations of antigen PD-L1 at equilibrium, reaction mixtures were performed in PBS (pH 7.4) with 1 mg/ml BSA. The measurements were performed with samples containing 200 pM of CDRI -007 and PD-L1 antigen in concentrations from 5 nM to 5 pM (two-fold serial dilutions). For trispecific CDR1-017, the measurements were performed using 1 nM of the antibody and two-fold serial dilutions from 100 nM to 100 pM for PD-L1 antigen. The equilibrium titration and kinetics data were fit to a 1:1 reversible binding model using KinExA Pro software (version 4.2.10; Sapidyne Instruments) to determine the K_{d}. The K_{d} value was predicted in the range of 21.7 to 42 pM for trispecific CDRI-007, and from 9.4 to 20.6 nM for trispecific CDRI -017. Overall, the K_{d} measurements by KinExA were lower than those determined by affinity characterization in solution by competition ELISA and some preliminary values obtained by SPR experiments (not described here). Affinity data from KinExA validated a difference in affinity for PD-L1 of about 1000-fold between CDR1-017 and CDRI-007 (WT).

The affinity of the trispecific antibody CDR1-007 for BCMA was further determined using MicroScale Thermophoresis (MST). Human BCMA was labelled with a fluorescent dye and kept at a constant concentration of 2 nM. The binding reactions were performed in PBS pH 7.4, 0.05% Tween-20, 1% BSA with samples containing 2 nM of fluorescently labeled BCMA and CDR1-007 in final concentrations from 500 nM to 15.3 pM (two-fold serial dilutions). The samples were analyzed on a Monolith NT.115 Pico at 25° C., with 5% LED power and 40% Laser power. The interaction between the trispecific antibody and BCMA showed a large amplitude (9 to 10 units) and a high signal to noise ratio (10.7 to 14.9), indicating optimal data quality. Binding affinity of the BCMA binding-arm was determined to be 8.5 to 9.9 nM in 2 different measurements. No sticking or aggregation effects were detected.

### Example 9

### Redirected T-Cell Cytotoxicity of H929 Myeloma Cells Induced by Trispecific Antibodies with Different Binding Affinities for PD-L1

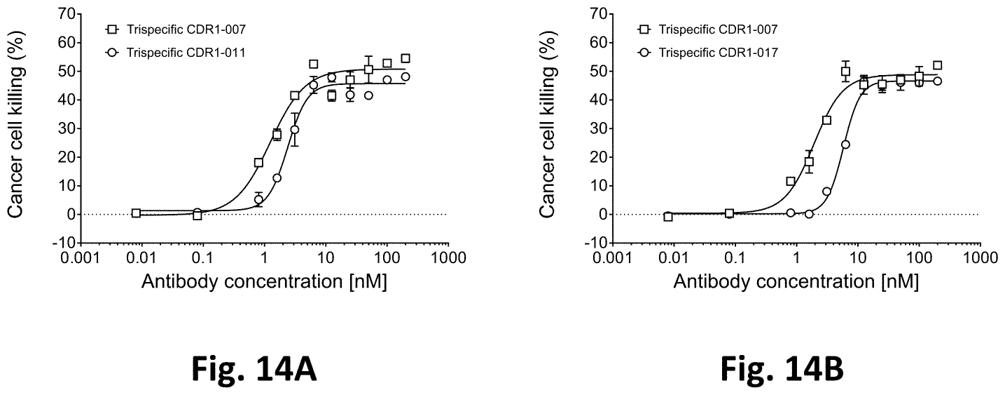

Trispecific antibodies with different binding affinities for PD-L1 were compared for the ability to induce T cell-mediated apoptosis of H929 human multiple myeloma cells. Trispecific antibodies CDR1-007, CDR1-011, and CDR1-017 at final concentrations ranging from 8 pM to 200 nM were incubated with isolated human CD3+ T cells and NCI-H929 human multiple myeloma cells as described in Example 5. As depicted in FIG. 14A and FIG. 14B, all trispecific antibodies induced potent lysis of H929 myeloma cells, and EC50 values were consistent with apparent affinities for PD-L1.

### Example 10

### Ex Vivo Assays with the Trispecific Antigen Binding Proteins

In vitro assays using multiple myeloma cell lines and PBMCs or purified T cells from normal blood donors had some limitations as they did not fully reflect the complexity and impact of the immune-suppressive environment of the bone marrow in multiple myeloma patients. Therefore, ex vivo assays were performed using bone marrow aspirates from multiple myeloma patients that mimic the situation in patients more closely than in vitro assays. For this, freshly acquired (not stored frozen) cells were prepared from the bone marrow aspirates collected from newly diagnosed, relapsed and multi-relapsed multiple myeloma patients. The resulting mononuclear cell suspensions were analyzed to determine the percentage of marker-positive cells via flow cytometry. The mononuclear cell suspensions were then placed in 384-well imaging plates in the presence of trispecific compounds and relevant controls in RPMI culture media with 10% FBS at 37° C. supplemented with 5% CO₂. After up to 72-hours incubation time, the cultures were followed by immunofluorescence staining and imaging using an automated microscopy platform as described in Nat Chem Biol. 2017 June; 13(6):681-690. All compounds were assayed at four concentrations and five technical replicates. The compounds evaluated in the image-based ex vivo testing were CDR1-007, CDR1-011 and CDR1-017, corresponding to high, mid and low affinity for PD-L1 (respectively), a bispecific control (CDR1-008), a combination of the bispecific antibody CDR1-008, the anti-PD-L1 inhibitor Avelumab (Expert Opin. Biol. Ther. 2017. 17(4): 515-523), and PBS as a negative control.

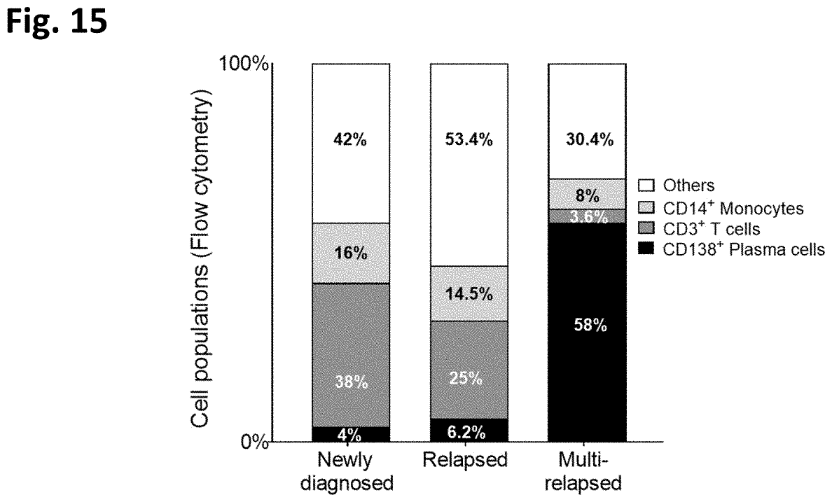

Different cell populations in the bone marrow samples were classified using fluorescently tagged antibodies against CD138, CD269 or CD319 for plasma cells, CD3 for T cells and CD14 for monocytes. The flow cytometry analysis of bone marrow aspirates for each patient sample revealed different percentages for the cell populations and a strong consistency between the plasma cell percentages of bone marrow sample (from 4% and up to 58%) and the state of disease for the multiple myeloma patients (FIG. 15).

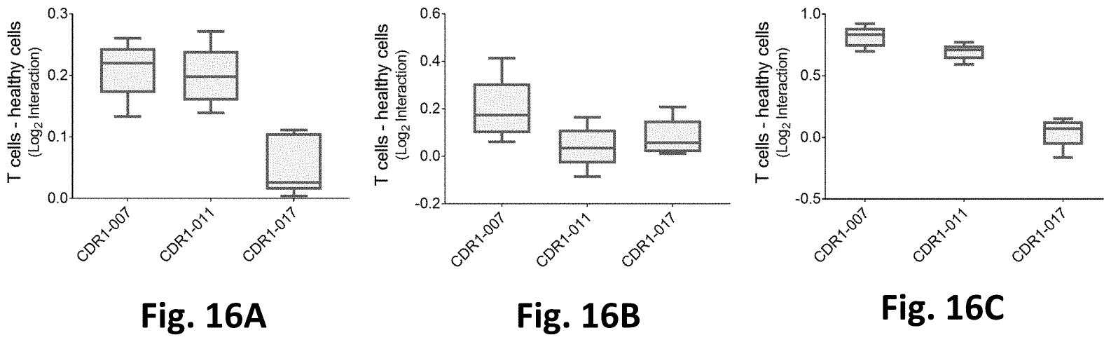

The ability of trispecific antibodies with different affinities for PD-L1 to avoid cross-linking T cells and normal cells was assessed ex-vivo. Imaging plates containing the patient samples and test compounds were incubated for 24 hours, CD3+ cells were identified using fluorescently tagged antibodies and normal cells based on DAPI-stain derived nucleus detection (not staining for extracellular markers CD3, CD138, CD269, CD319 or CD14). Interactions of CD3+ cells with normal cells were evaluated based on an interaction score as described in Nat. Chem. Biol. 2017 June; 13(6): 681-690. Increased cell-cell interactions were observed between the CD3+ cells and normal cells incubated with CDR1-007 and CDR1-011 in samples from newly diagnosed (FIG. 16A), relapsed (FIG. 16B), and multi-relapsed (FIG. 16C) multiple myeloma patients. Importantly, CDR1-017 did not increase interactions of CD3+ cells with normal cells, indicating that reduced affinity for PD-L1 successfully reduced binding of the trispecific CDR1-017 to normal cells expressing only PD-L1.

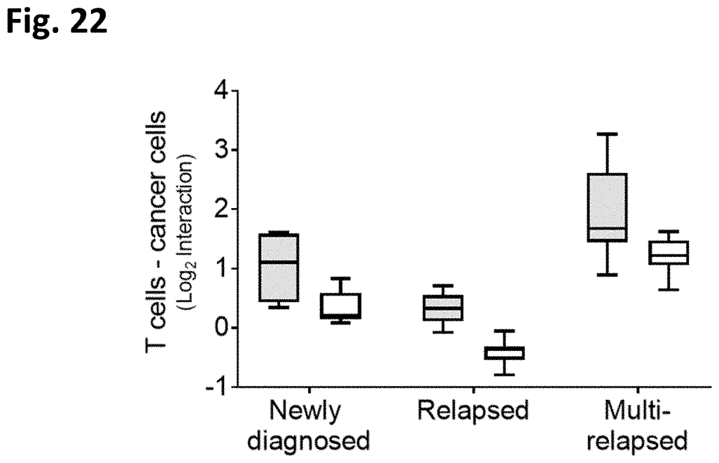

Next, the CDR1-017 trispecific antibody was evaluated for the ability to redirect CD3+ T cells to the target cell population staining for CD138, CD269, or CD319. As depicted in FIG. 22, the trispecific antibody CDR1-017 (filled boxes) increased interactions between T cells and plasma cells in the samples from the different multiple myeloma patients more efficiently than the bispecific antibody CDR1-008 (empty boxes).

These results suggested that the additional binding site for PD-L1 in the trispecific antibody CDR1-017 contributed to a more efficient redirection of T cells compared with the bispecific antibody CDR1-008.

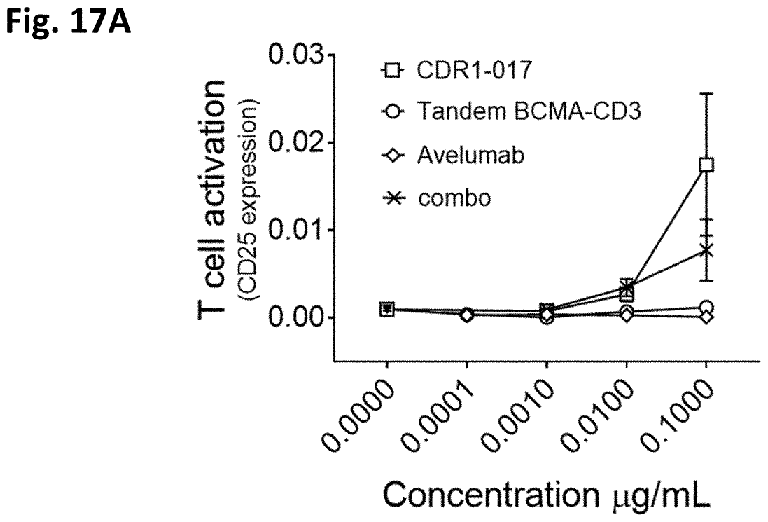

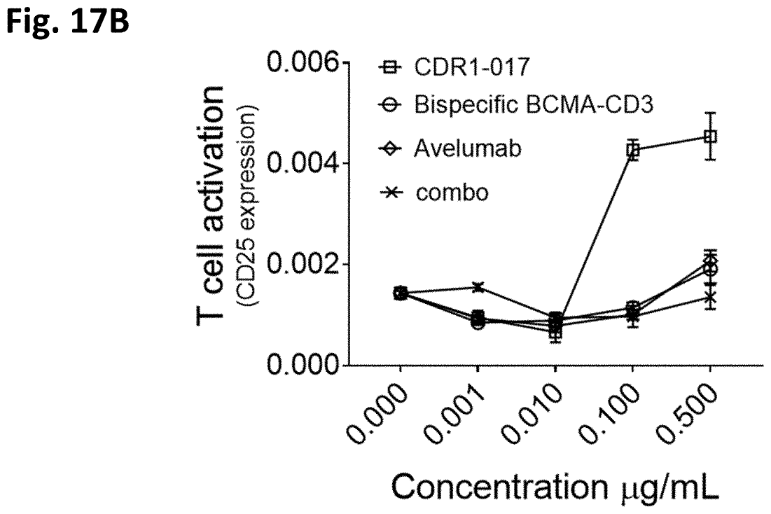

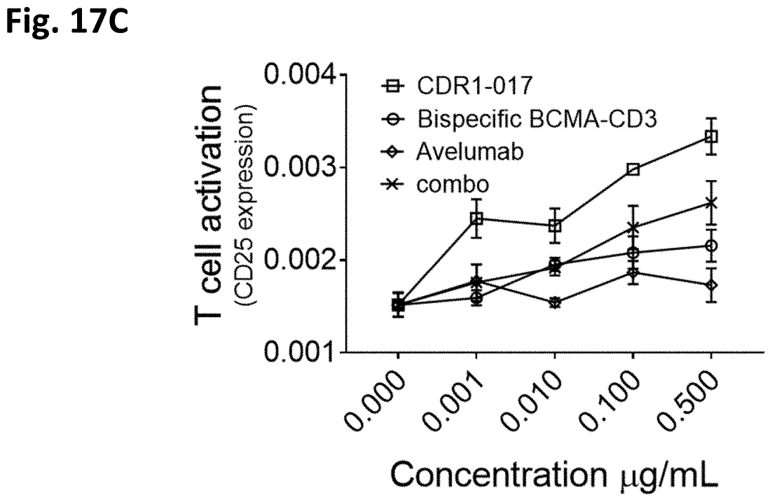

In a different readout, T cell activation was assessed by quantifying CD25 expression intensity on CD3+ population in the presence of test compound. FIG. 17A-17C showed that CDR1-017 potently activated T cells from the newly diagnosed, relapsed and multi-relapsed patients, regardless of the different ratios of cell populations. Indeed, CDR1-017 significantly surpassed the level of T cell activation achieved with the BCMA/CD3 bispecific antibody, as well as the T cell activation obtained through combination of anti-PD-L1 and the BCMA/CD3 bispecific.

This experiment demonstrated that CDR1-017 efficiently redirected T cells to cancer cells and simultaneously induced local activation of T cells via PD-1/PD-L1 blockade while avoiding a potential ‘antigen sink’ created by cells expressing PD-L1. Together, these results established trispecific antibodies targeting CD3 and PD-L1 along with a tumor antigen as a viable strategy for directing the synergistic benefits of combination therapy specifically toward tumor cells.

### Example 11

### Thermal Stability Assessment

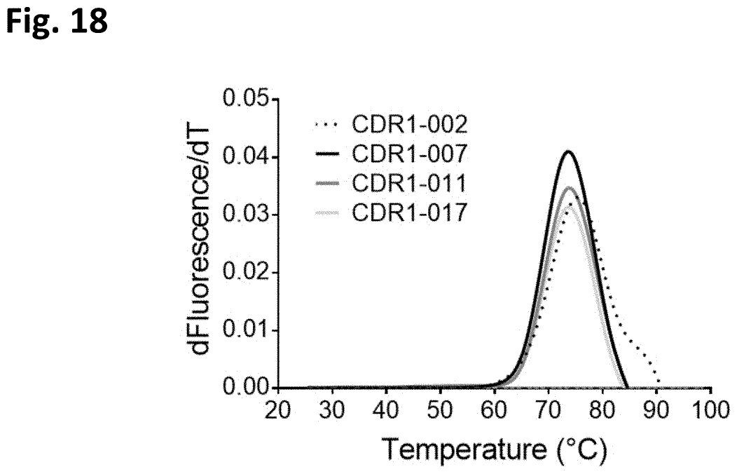

Thermal unfolding experiments with the antibodies of the invention were performed using two methods: 1) conventional differential scanning fluorimetry (DSF); and 2) nanoDSF. Briefly, for DSF experiments, a linear temperature ramp was applied to unfold protein samples and protein unfolding was detected based on the interactions of a fluorescent dye (SYPRO® Orange) with hydrophobic patches which became exposed to the solvent upon heating. Representative data for the thermal unfolding experiments by DSF are shown in FIG. 18. Samples were measured at concentrations ranging from 2 to 3 μM in 10 mM sodium phosphate (pH 6.5) and 150 mM NaCl buffer using a temperature gradient from 25 to 98° C. with a heating speed of 3° C./minute. CDR1-007, CDR1-011 and CDR1-017 showed high stability with transitions of unfolding at 74° C. For nanoDSF experiments, seven trispecific antibodies and two Fabs were measured at concentrations ranging from 1.6 to 5 μM and were submitted to a temperature gradient of 20-95° C. with heating speed of 1° C/minute using a Prometheus NT. Plex (Nanotemper). Comparison of Tm data from nanoDSF and μDSC data showed a good agreement between the methods where a single unfolding event was detected for CDR1-007, CDR1-0011 and CDR1-017. The higher Tm determined in DSF was attributed to the faster scan rate.

### Example 12

### Stability Studies with Trispecific Antibodies

To assess the oligomerization/fragmentation propensity of trispecific antibodies, CDR1-007, CDR1-011 and CDR1-017 were concentrated to 10 mg/mL in formulation buffer (10 mM phosphate, 140 mM NaCl) pH 6.5, and incubated for 2 weeks at 37° C. Samples were analyzed before and after 14 days incubation using size-exclusion chromatography for the quantification of the monomeric protein, aggregates and low molecular weight species. Monomers were resolved from nonmonomeric species by HPLC on a TSKgel Super SW2000 column (TOSOH Bioscience). The percentage of monomeric protein was calculated as the area of the monomer peak divided by the total area of all product peaks.

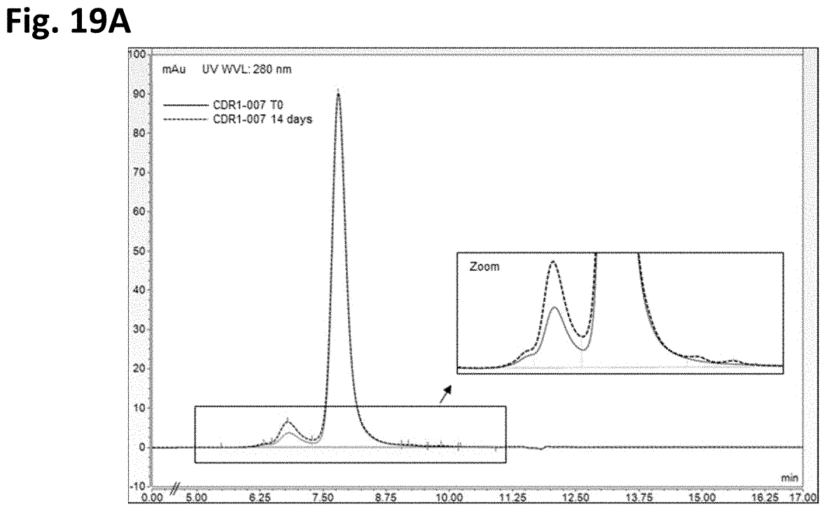

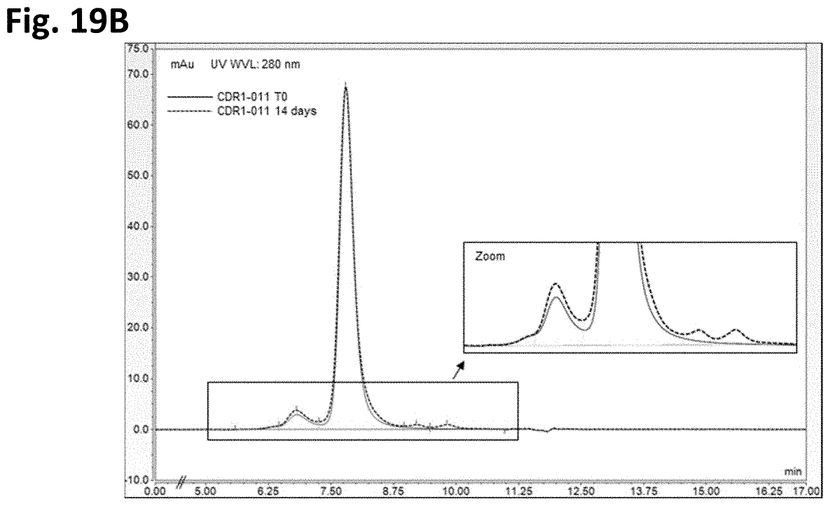

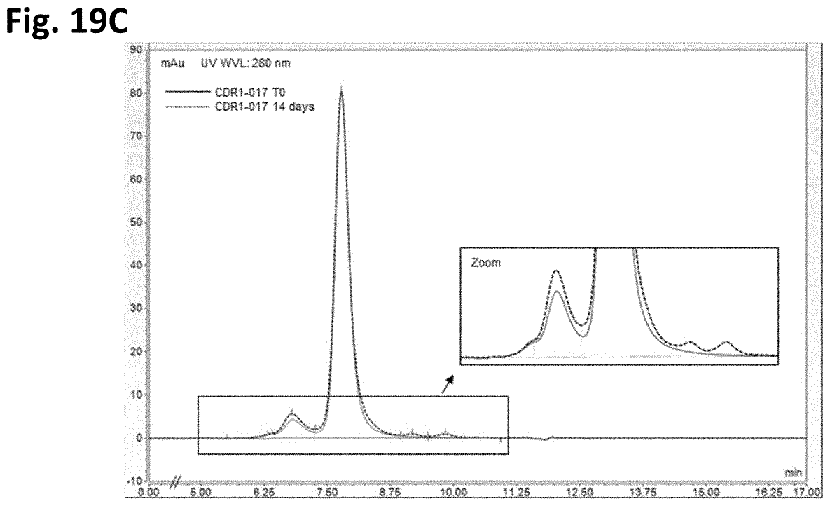

All trispecific samples showed good stability in non-optimized buffer after 2 weeks incubation at 37° C. FIG. 19 depicts size exclusion chromatography analysis for CDR1-007 (FIG. 19A), CDR1-011 (FIG. 19B), and CDR1-017 (FIG. 19C). The main peak was assigned to the monomeric protein eluted from the column after approximately 7.8 minutes (consistent with the expected elution time), and good resolution between monomer and the aggregate peaks as well as the fragments was obtained. The monomer content of the trispecific protein samples before incubation was approximately 94% for CDR1-007 and CDR1-011 and 92% for CDR1-017. Monomer loss of the samples in non-optimized buffer after 2 weeks incubation at 37° C. was about 4% for all samples. Additional peaks were assigned to defined molecular weight aggregates and low molecular-weight species.

### Example 13

### Ability of Trispecific Antibodies with Specificity for Different TAAs to Activate T Cells Upon Engagement of Cancer Cell Lines

Three trispecific antibodies binding to different tumor associated antigens (TAAs) were evaluated their ability to induce IL-2 cytokine production in isolated human CD3+cells upon engagement of relevant cancer cell lines. The antibodies CDR1-061, with specificity for CD3, PD-L1 and CD19, and CDR1-08, with specificity for CD3, PD-L1 and HER2, were compared head-to-head to their respective bispecific controls (CDR1-063 and CDR1-083) for their ability to activate T cells measured as a function of IL-2 production. The trispecific antibody CDR1-007 with specificity for BCMA and bispecific control CDR1-008 were also included as a reference.

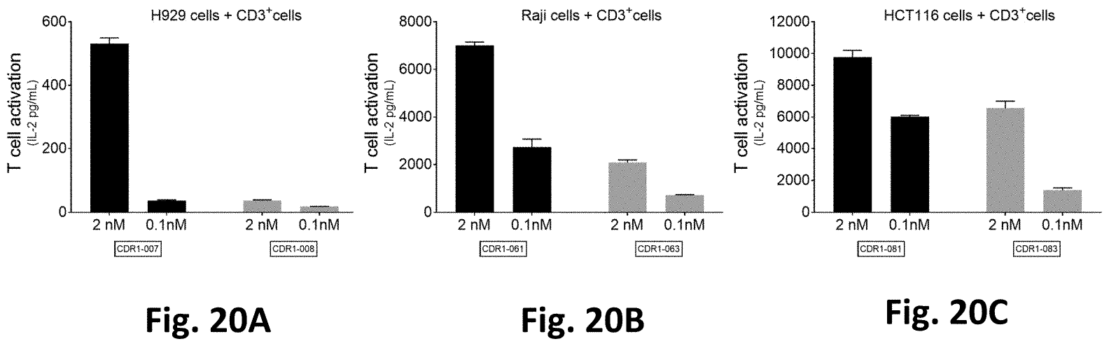

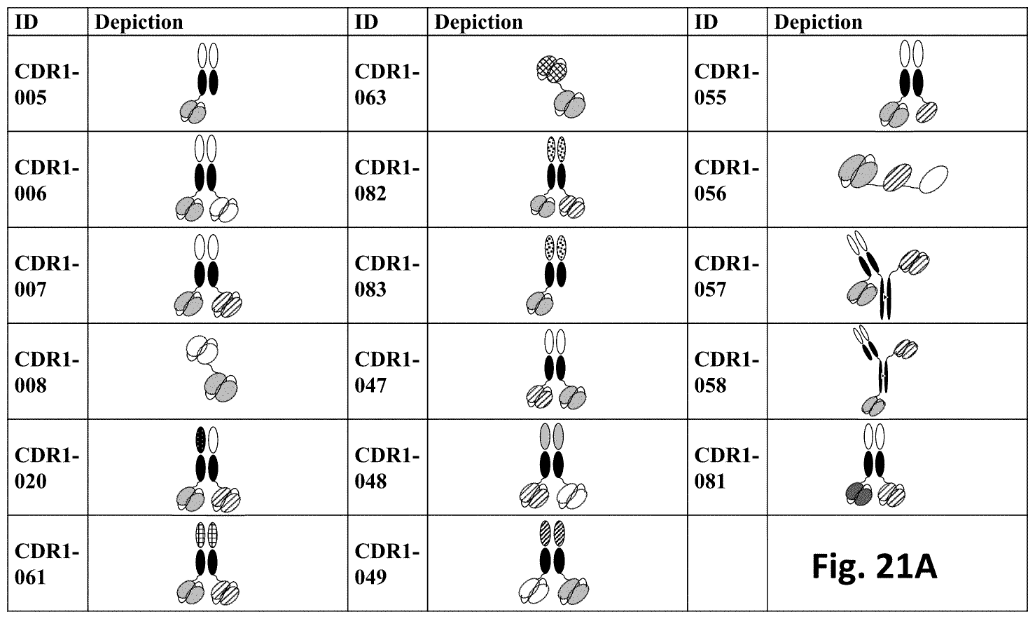

Briefly, human CD3+ T cells were isolated from PBMCs as described in Examples 4 and 5, and 1×10⁵ isolated CD3+ T cells (effector) were co-incubated with NCI-H929 human multiple myeloma cells, B-cell lymphoma line Raji (ATCC® CCL-86™) and a human colorectal carcinoma cell line HCT116 (ATCC® CCL-247™) at effector to target cell ratio of 5:1, in the presence of 0.1 nM and 2 nM antibody concentrations. FIG. 20 shows IL-2 measured in the supernatants of T cells co-cultured with H929 multiple myeloma cells (FIG. 20A), Raji lymphoma cells (FIG. 20B), and HCT116 cells (FIG. 20C) in presence of the different trispecific antibodies and their respective bispecific controls. The results of these experiments show that all three trispecific antibodies induced production of IL-2 cytokine by the isolated human T cells more efficiently than the bispecific controls. This indicates that this approach can be effectively used in several malignancies to rescue PD-L1 mediated inhibition of human T cell activation.

1. A trispecific antigen binding protein comprising:
a) a first binding domain capable of binding to a cell surface protein of a tumor cell;
b) a second binding domain capable of binding to a cell surface immune checkpoint protein of the tumor cell; and
c) a third binding domain capable of binding to a cell surface protein of an immune cell,
wherein:
the first binding domain binds to a cell surface protein of a tumor cell with reduced affinity to suppress binding to non-tumor cells or a soluble form of the cell surface protein; and/or
the second binding domain binds to a cell surface immune checkpoint protein of the tumor cell with reduced affinity to suppress binding to non-tumor cells.

2. (canceled)

3. The trispecific antigen binding protein of claim 1, wherein:
the cell surface protein of the tumor cell is selected from the group consisting of BCMA, CD19, CD20, CD33, CD123, CEA, LMP1, LMP2, PSMA, FAP, and HER2;
the cell surface immune checkpoint protein of the tumor cell is selected from the group consisting of CD40, CD47, CD80, CD86, GAL9, PD-L1, and PD-L2; and/or
the third binding domain binds CD3, TCRα, TCRβ, CD16, NKG2D, CD89, CD64, or CD32a on the immune cell.

4. The trispecific antigen binding protein of claim 1, wherein:
the first binding domain binds BCMA on the tumor cell;
the second binding domain binds PD-L1 on the tumor cell; and/or
the third binding domain binds to CD3 on the immune cell.

5-8. (cancel)

9. The trispecific antigen binding protein of claim 1, wherein:
the first binding domain affinity is between about 1 nM to about 100 nM or about 10 nM to about 80 nM; and/or
the second binding domain affinity is between about 1 nM to about 100 nM or about 10 nM to about 80 nM.

10-12. (cancel)

13. The trispecific antigen binding protein of claim 1, wherein:
the first and second binding domain bind target antigens on the same cell to increase binding avidity;
the first binding domain comprises low affinity to the cell surface protein of the tumor cell to reduce crosslinking to healthy cells or a soluble form of the cell surface protein;
the second binding domain comprises low affinity to the cell surface immune checkpoint protein of the tumor cell to reduce crosslinking to healthy cells;
the first and second binding domain each comprise low affinity to the target antigens of the tumor cell, wherein the trispecific antigen binding protein comprises enhanced crosslinking to the tumor cell relative to crosslinking to healthy cells;
the first and second binding domain bind target antigens on the same cell to reduce off-target binding to healthy tissue;
the first, second, and third binding domains have reduced off-target binding;
the cell surface protein of a tumor cell is absent or has limited expression on healthy cells relative to tumor cells; and/or
the second binding domain has low affinity to the cell surface immune checkpoint protein of the tumor cell to reduce checkpoint inhibition on healthy cells.

14-20. (cancel)

21. The trispecific antigen binding protein of claim 1, wherein the first, second, and third binding domains comprise an antibody, optionally wherein:
the first, second, and third binding domains comprise an scFv, an sdAb, or a Fab fragment;
the second binding domain is monovalent;
the third binding domain is monovalent;
the first, second, and third binding domains are joined together by one or more linkers;
the trispecific antigen binding protein has a molecular weight of about 75 kDa to about 100 kDa; and/or
the trispecific antigen binding protein has increased serum half-life relative to an antigen binding protein with a molecular weight of ≤about 60 kDa.

22-27. (cancel)

28. A trispecific antigen binding protein comprising:
a) a first binding domain capable of binding to a cell surface protein of a tumor cell;
b) a second binding domain capable of binding to PD-L1 on the surface of the tumor cell; and
c) a third binding domain capable of binding to CD3 on the surface of a T cell, wherein the first and second binding domains bind to a cell surface protein of a tumor cell and to PD-L1 with reduced affinity to suppress binding to non-tumor cells.

29. The trispecific antigen binding protein of claim 28, wherein the cell surface protein of the tumor cell is selected from the group consisting of BCMA, CD19, CD20, CD33, CD123, CEA, LMP1, LMP2, PSMA, FAP, and HER2.

30. (canceled)

31. The trispecific antigen binding protein of claim 28, wherein:
the first binding domain affinity is between about 1 nM to about 100 nM or about 10 nM to about 80 nM;
the second binding domain affinity is between about 1 nM to about 100 nM or about 1 nM to about 80 nM;
the first and second binding domain bind target antigens on the same cell to increase binding avidity;
the first binding domain comprises low affinity to the cell surface protein of the tumor cell to reduce crosslinking to healthy cells or a soluble form of the cell surface protein;
the second binding domain comprises low affinity to PD-L1 on the surface of the tumor cell to reduce crosslinking to healthy cells;
the first and second binding domain each comprise low affinity to the target antigens of the tumor cell, wherein the trispecific antigen binding protein comprises enhanced crosslinking to the tumor cell relative to crosslinking to healthy cells;
the first and second binding domain bind target antigens on the same cell to reduce off-target binding to healthy tissue;
the first, second, and third binding domains have reduced off-target binding;
the cell surface protein of a tumor cell is absent or has limited expression on healthy cells relative to tumor cells; and/or
the second binding domain has low affinity to PD-L1 on the surface of the tumor cell to reduce checkpoint inhibition on healthy cells.

32-42. (cancel)

43. The trispecific antigen binding protein of claim 28, wherein the first, second, and third binding domains comprise an antibody, optionally wherein:
the first, second, and third binding domains comprise an scFv, an sdAb, or a Fab fragment the second binding domain is monovalent;
the third binding domain is monovalent;
the first, second, and third binding domains are joined together by one or more linkers;
the trispecific antigen binding protein has a molecular weight of about 75 kDa to about 100 kDa; and/or
the trispecific antigen binding protein has increased serum half-life relative to an antigen binding protein with a molecular weight of ≤about 60 kDa.

44-50. (cancel)

50. A trispecific antigen binding protein comprising two different chains, wherein:
a) one chain comprises at least one heavy chain (Fd fragment) of a Fab fragment linked to at least one additional binding domain; and
b) the other chain comprises at least one light chain (L) of a Fab fragment linked to at least one additional binding domain,
wherein the Fab domain optionally serves as a specific heterodimerization scaffold to which the additional binding domains are optionally linked, and the binding domains have different specificities.

51. The trispecific antigen binding protein of claim 50, wherein the additional binding domains are an scFv or an sdAb.

52. The trispecific antigen binding protein of claim 50, wherein the trispecific binding protein comprises:
i) a first binding domain capable of binding to a cell surface protein of a tumor cell;
ii) a second binding domain capable of binding to a cell surface immune checkpoint protein of the tumor cell; and
iii) a third binding domain capable of binding to a cell surface protein of an immune cell.

53. The trispecific antigen binding protein of claim 50, wherein the additional binding domains are linked to the N terminus or C terminus of the heavy chain or light chain of the Fab fragment.

54. A method of treating cancer in a subject, comprising administering to the subject a therapeutically effective amount of the trispecific antigen binding protein of claim 1, optionally wherein the cancer is selected from the group consisting of multiple myeloma, acute myeloid leukemia.

55-63. (cancel)

64. An ex vivo method of identifying antigen binding domains capable of one or both of binding to a cell surface protein of a tumor cell and a cell surface immune checkpoint protein of a tumor cell, the method comprising:
a) isolating peripheral blood mononuclear cells (PBMCs) or bone marrow plasma cells (PCs) and autologous bone marrow infiltrating T cells from a patient suffering from cancer;
b) contacting the PBMCs or PCs with a panel of trispecific antigen binding proteins, wherein a first domain of the trispecific antigen binding protein binds to CD3 on T cells and a second domain of the trispecific antigen binding protein binds to a cell surface protein of a tumor cell and/or a cell surface immune checkpoint protein of a tumor cell;
c) determining drug killing of cancer cells by measuring one or more trispecific antigen binding protein effects on immune-mediated cancer cell killing; and
d) selecting the trispecific antigen binding proteins based on their ability to induce immune-mediated cancer cell killing.

65. The ex vivo method of claim 64, wherein a trispecific antigen binding protein effect on immune-mediated cancer cell killing comprises lactate dehydrogenase (LDH) release.

66. The ex vivo method of claim 64, wherein a trispecific antigen binding protein effect on immune-mediated cancer cell killing comprises number of depleted target cancer cells.


SEQUENCE LISTING (71 sequences)

SEQ ID NO: 1 - protein, 481 aa, Artificial Sequence
  source: /note=“Description of Artificial Sequence Synthetic polypeptide”
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEIVMTQSPSTLSASVGDRVIITCQSSQSVYGNIWMAWYQQKPGRAPKLLIYQASKLASGVPSRFSGSGSGAEFTLTISSLQPDDSATYYCQGNFNTGDRYAFGQGTKLTVLGGGGGSGGGGSGGGGSGGGGSEVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSS

SEQ ID NO: 2 - protein, 475 aa, Artificial Sequence
  source: /note=“Description of Artificial Sequence Synthetic polypeptide”
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEIVMTQSPSTLSASVGDRVIITCQSSQSVYGNIWMAWYQQKPGRAPKLLIYQASKLASGVPSRFSGSGSGAEFTLTISSLQPDDSATYYCQGNFNTGDRYAFGQGTKLTVLGGGGGSGGGGSGGGGSGGGGSEVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSS

SEQ ID NO: 3 - protein, 480 aa, Artificial Sequence
  source: /note=“Description of Artificial Sequence Synthetic polypeptide”
EVQLVESGGGLVQPGGSLRLSCTASGFTISRSYWICWVRQAPGKGLEWVGCIYGDNDITPLYANWAKGRFTISRDTSKNTVYLQMNSLRAEDTAVYYCARLGYADYAYDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEIVMTQSPSTLSASVGDRVIITCQASEIIHSWLAWYQQKPGKAPKLLIYLASTLASGVPSRFSGSGSGAEFTLTISSLQPDDSATYYCQNVYLASTNGANFGQGTKLTVLGGGGGSGGGGSGGGGSGGGGSEVQLVESGGGSVQPGGSLRLSCTASGFSLTDYYYMTWVRQAPGKGLEWVGFIDPDDDPYYATWAKGRFTISRDNSKNTLYLQMNSLRAEDTATYYCAGGDHNSGWGLDIWGQGTTVTVSS

SEQ ID NO: 4 - protein, 474 aa, Artificial Sequence
  source: /note=“Description of Artificial Sequence Synthetic polypeptide”
EIVMTQSPSTLSASVGDRVIITCQSSQSVYGNIWMAWYQQKPGRAPKLLIYQASKLASGVPSRFSGSGSGAEFTLTISSLQPDDFATYYCQGNFNTGDRYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEIVMTQSPSTLSASVGDRVIITCQASEIIHSWLAWYQQKPGKAPKLLIYLASTLASGVPSRFSGSGSGAEFTLTISSLQPDDSATYYCQNVYLASTNGANFGQGTKLTVLGGGGGSGGGGSGGGGSGGGGSEVQLVESGGGSVQPGGSLRLSCTASGFSLTDYYYMTWVRQAPGKGLEWVGFIDPDDDPYYATWAKGRFTISRDNSKNTLYLQMNSLRAEDTATYYCAGGDHNSGWGLDIWGQGTTVTVSS

SEQ ID NO: 5 - protein, 714 aa, Artificial Sequence
  source: /note=“Description of Artificial Sequence Synthetic polypeptide”
EVQLVESGGGLVQPGGSLRLSCTASGFTISRSYWICWVRQAPGKGLEWVGCIYGDNDITPLYANWAKGRFTISRDTSKNTVYLQMNSLRAEDTAVYYCARLGYADYAYDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSEVQLVESGGGLVQPGGSLRLSCTASGFTISRSYWICWVRQAPGKGLEWVGCIYGDNDITPLYANWAKGRFTISRDTSKNTVYLQMNSLRAEDTAVYYCARLGYADYAYDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEIVMTQSPSTLSASVGDRVIITCQASEIIHSWLAWYQQKPGKAPKLLIYLASTLASGVPSRFSGSGSGAEFTLTISSLQPDDSATYYCQNVYLASTNGANFGQGTKLTVLGGGGGSGGGGSGGGGSGGGGSEVQLVESGGGSVQPGGSLRLSCTASGFSLTDYYYMTWVRQAPGKGLEWVGFIDPDDDPYYATWAKGRFTISRDNSKNTLYLQMNSLRAEDTATYYCAGGDHNSGWGLDIWGQGTTVTVSS

SEQ ID NO: 6 - protein, 218 aa, Artificial Sequence
  source: /note=“Description of Artificial Sequence Synthetic polypeptide”
EIVMTQSPSTLSASVGDRVIITCQSSQSVYGNIWMAWYQQKPGRAPKLLIYQASKLASGVPSRFSGSGSGAEFTLTISSLQPDDFATYYCQGNFNTGDRYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 714 aa, Artificial Sequence
  source: /note=“Description of Artificial Sequence Synthetic polypeptide”
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEIVMTQSPSTLSASVGDRVIITCQSSQSVYGNIWMAWYQQKPGRAPKLLIYQASKLASGVPSRFSGSGSGAEFTLTISSLQPDDSATYYCQGNFNTGDRYAFGQGTKLTVLGGGGGSGGGGSGGGGSGGGGSEVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSS

SEQ ID NO: 8 - protein, 217 aa, Artificial Sequence
  source: /note=“Description of Artificial Sequence Synthetic polypeptide”
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 220 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 10 - protein, 477 aa, Artificial Sequence
  source: /note=“Description of Artificial Sequence Synthetic polypeptide”
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 11 - protein, 473 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 12 - protein, 479 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEIVMTQSPSTLSASVGDRVIITCQASEDIYSLLAWYQQKPGKAPKLLIYDASDLASGVPSRFSGSGSGAEFTLTISSLQPDDSATYYCQGNYGSSSSSSYGAVFGQGTKLTVLGGGGGSGGGGSGGGGSGGGGSEVQLVESGGGSVQPGGSLRLSCTVSGIDLSSYTMGWVRQAPGKGLEWVGIISSGGRTYYASWAKGRFTISRDTSKNTVYLQMNSLRAEDTATYYCARGRYTGYPYYFALWGQGTTVTVSS

SEQ ID NO: 13 - protein, 507 aa, Artificial Sequence
  source: /note=“Description of Artificial Sequence Synthetic polypeptide”
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 14 - protein, 477 aa, Artificial Sequence
  source: /note=“Description of Artificial Sequence Synthetic polypeptide”
EIVMTQSPSTLSASVGDRVIITCQSSQSVYGNIWMAWYQQKPGRAPKLLIYQASKLASGVPSRFSGSGSGAEFTLTISSLQPDDFATYYCQGNFNTGDRYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 15 - protein, 479 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 16 - protein, 477 aa, Artificial Sequence
  source: /note=“Description of Artificial Sequence Synthetic polypeptide”
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEIVMTQSPSTLSASVGDRVIITCQASEDIYSLLAWYQQKPGKAPKLLIYDASDLASGVPSRFSGSGSGAEFTLTISSLQPDDSATYYCQGNYGSSSSSSYGAVFGQGTKLTVLGGGGGSGGGGSGGGGSGGGGSEVQLVESGGGSVQPGGSLRLSCTVSGIDLSSYTMGWVRQAPGKGLEWVGIISSGGRTYYASWAKGRFTISRDTSKNTVYLQMNSLRAEDTATYYCARGRYTGYPYYFALWGQGTTVTVSS

SEQ ID NO: 17 - protein, 481 aa, Artificial Sequence
  source: /note=“Description of Artificial Sequence Synthetic polypeptide”
EVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 18 - protein, 474 aa, Artificial Sequence
  source: /note=“Description of Artificial Sequence Synthetic polypeptide”
AVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEIVMTQSPSTLSASVGDRVIITCQASEDIYSLLAWYQQKPGKAPKLLIYDASDLASGVPSRFSGSGSGAEFTLTISSLQPDDSATYYCQGNYGSSSSSSYGAVFGQGTKLTVLGGGGGSGGGGSGGGGSGGGGSEVQLVESGGGSVQPGGSLRLSCTVSGIDLSSYTMGWVRQAPGKGLEWVGIISSGGRTYYASWAKGRFTISRDTSKNTVYLQMNSLRAEDTATYYCARGRYTGYPYYFALWGQGTTVTVSS

SEQ ID NO: 19 - protein, 482 aa, Artificial Sequence
  source: /note=“Description of Artificial Sequence Synthetic polypeptide”
EVQLVESGGGSVQPGGSLRLSCTVSGIDLSSYTMGWVRQAPGKGLEWVGIISSGGRTYYASWAKGRFTISRDTSKNTVYLQMNSLRAEDTATYYCARGRYTGYPYYFALWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 20 - protein, 473 aa, Artificial Sequence
  source: /note=“Description of Artificial Sequence Synthetic polypeptide”
EIVMTQSPSTLSASVGDRVIITCQASEDIYSLLAWYQQKPGKAPKLLIYDASDLASGVPSRFSGSGSGAEFTLTISSLQPDDSATYYCQGNYGSSSSSSYGAVFGQGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 21 - protein, 353 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSQVQLVESGGGLVQPGGSLRLSCAASGKMSSRRCMAWFRQAPGKGLERVAKLLTTSGSTYLADSVKGRFTISRDNSKNTVYLQMNSLRAEDTAVYYCAADSFEDPTCTLVTSSGAFQYWGQGTLVTVSS

SEQ ID NO: 22 - protein, 515 aa, Artificial Sequence
  source: /note=“Description of Artificial Sequence Synthetic polypeptide”
AVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSSGGGGSQVQLVESGGGLVQPGGSLRLSCAASGKMSSRRCMAWFRQAPGKGLERVAKLLTTSGSTYLADSVKGRFTISRDNSKNTVYLQMNSLRAEDTAVYYCAADSFEDPTCTLVTSSGAFQYWGQGTLVTVSSGGGGSQVQLVESGGGLVQPGGSLRLSCAASGFTLDYYAIGWFRQAPGKEREGVSCISRSDGSTYYADSVKGRFTISRDNAKNTVYLQMNSLKPEDTAVYYCAAAGADCSGYLRDYEFWGQGTLVTVSS

SEQ ID NO: 23 - protein, 447 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 491 aa, Artificial Sequence
  source: /note=“Description of Artificial Sequence Synthetic polypeptide”
EIVMTQSPSTLSASVGDRVIITCQASEDIYSLLAWYQQKPGKAPKLLIYDASDLASGVPSRFSGSGSGAEFTLTISSLQPDDSATYYCQGNYGSSSSSSYGAVFGQGTKLTVLGGGGGSGGGGSGGGGSGGGGSEVQLVESGGGSVQPGGSLRLSCTVSGIDLSSYTMGWVRQAPGKGLEWVGIISSGGRTYYASWAKGRFTISRDTSKNTVYLQMNSLRAEDTATYYCARGRYTGYPYYFALWGQGTTVTVSSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 706 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 26 - protein, 218 aa, Artificial Sequence
  source: /note=“Description of Artificial Sequence Synthetic polypeptide”
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 486 aa, Artificial Sequence
  source: /note=“Description of Artificial Sequence Synthetic polypeptide”
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEIVMTQSPSTLSASVGDRVIITCQASEDIYSLLAWYQQKPGKAPKLLIYDASDLASGVPSRFSGSGSGAEFTLTISSLQPDDSATYYCQGNYGSSSSSSYGAVFGQGTKLTVLGGGGGSGGGGSGGGGSGGGGSEVQLVESGGGSVQPGGSLRLSCTVSGIDLSSYTMGWVRQAPGKGLEWVGIISSGGRTYYASWAKGRFTISRDTSKNTVYLQMNSLRAEDTATYYCARGRYTGYPYYFALWGQGTTVTVSS

SEQ ID NO: 28 - protein, 476 aa, Artificial Sequence
  source: /note=“Description of Artificial Sequence Synthetic polypeptide”
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 29 - protein, 514 aa, Artificial Sequence
  source: /note=“Description of Artificial Sequence Synthetic polypeptide”
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSSGGGGSGGGGSGGGGSGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLG

SEQ ID NO: 30 - protein, 482 aa, Artificial Sequence
  source: /note=“Description of Artificial Sequence Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEIVMTQSPSTLSASVGDRVIITCQASEDIYSLLAWYQQKPGKAPKLLIYDASDLASGVPSRFSGSGSGAEFTLTISSLQPDDSATYYCQGNYGSSSSSSYGAVFGQGTKLTVLGGGGGSGGGGSGGGGSGGGGSEVQLVESGGGSVQPGGSLRLSCTVSGIDLSSYTMGWVRQAPGKGLEWVGIISSGGRTYYASWAKGRFTISRDTSKNTVYLQMNSLRAEDTATYYCARGRYTGYPYYFALWGQGTTVTVSS

SEQ ID NO: 31 - protein, 473 aa, Artificial Sequence
  source: /note=“Description of Artificial Sequence Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 32 - protein, 223 aa, Artificial Sequence
  source: /note=“Description of Artificial Sequence Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 33 - protein, 117 aa, Artificial Sequence
  source: /note=“Description of Artificial Sequence Synthetic polypeptide”
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 34 - protein, 111 aa, Artificial Sequence
  source: /note=“Description of Artificial Sequence Synthetic polypeptide”
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 35 - protein, 120 aa, Artificial Sequence
  source: /note=“Description of Artificial Sequence Synthetic polypeptide”
EVQLVESGGGLVQPGGSLRLSCTVSGIDLSSYTMGWVRQAPGKGLEWVGIISSGGRTYYASWAKGRFTISRDTSKNTVYLQMNSLRAEDTAVYYCARGRYTGYPYYFALWGQGTLVTVSS

SEQ ID NO: 36 - protein, 114 aa, Artificial Sequence
  source: /note=“Description of Artificial Sequence Synthetic polypeptide”
EIVMTQSPSTLSASVGDRVIITCQASEDIYSLLAWYQQKPGKAPKLLIYDASDLASGVPSRFSGSGSGAEFTLTISSLQPDDFATYYCQGNYGSSSSSSYGAVFGQGTKLTVLG

SEQ ID NO: 37 - protein, 123 aa, Artificial Sequence
  source: /note=“Description of Artificial Sequence Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 38 - protein, 106 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 39 - protein, 125 aa, Artificial Sequence
  source: /note=“Description of Artificial Sequence Synthetic polypeptide”
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 40 - protein, 108 aa, Artificial Sequence
  source: /note=“Description of Artificial Sequence Synthetic polypeptide”
AVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 41 - protein, 125 aa, Artificial Sequence
  source: /note=“Description of Artificial Sequence Synthetic polypeptide”
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 42 - protein, 109 aa, Artificial Sequence
  source: /note=“Description of Artificial Sequence Synthetic polypeptide”
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 43 - protein, 119 aa, Artificial Sequence [sequence omitted]

SEQ ID NO: 44 - protein, 106 aa, Artificial Sequence [sequence omitted]

SEQ ID NO: 45 - protein, 5 aa, Artificial Sequence
  source: /note=“Description of Artificial Sequence Synthetic peptide”
GGGGS

SEQ ID NO: 46 - protein, 20 aa, Artificial Sequence
  source: /note=“Description of Artificial Sequence Synthetic peptide”
GGGGSGGGGSGGGGSGGGGS

SEQ ID NO: 47 - protein, 5 aa, Artificial Sequence
  source: /note=“Description of Artificial Sequence Synthetic peptide”
DGGGS

SEQ ID NO: 48 - protein, 5 aa, Artificial Sequence
  source: /note=“Description of Artificial Sequence Synthetic peptide”
TGEKP

SEQ ID NO: 49 - protein, 4 aa, Artificial Sequence
  source: /note=“Description of Artificial Sequence Synthetic peptide”
GGRR

SEQ ID NO: 50 - protein, 25 aa, Artificial Sequence
  source: /note=“Description of Artificial Sequence Synthetic peptide”
  SITE (1)..(25): /note=“This sequence may encompass 1-5 ′Gly Gly Gly Gly Ser′ repeating units”
GGGGSGGGGSGGGGSGGGGSGGGGS

SEQ ID NO: 51 - protein, 14 aa, Artificial Sequence
  source: /note=“Description of Artificial Sequence Synthetic peptide”
EGKSSGSGSESKVD

SEQ ID NO: 52 - protein, 18 aa, Artificial Sequence
  source: /note=“Description of Artificial Sequence Synthetic peptide”
KESGSVSSEQLAQFRSLD

SEQ ID NO: 53 - protein, 8 aa, Artificial Sequence
  source: /note=“Description of Artificial Sequence Synthetic peptide”
GGRRGGGS

SEQ ID NO: 54 - protein, 9 aa, Artificial Sequence
  source: /note=“Description of Artificial Sequence Synthetic peptide”
LRQRDGERP

SEQ ID NO: 55 - protein, 12 aa, Artificial Sequence
  source: /note=“Description of Artificial Sequence Synthetic peptide”
LRQKDGGGSERP

SEQ ID NO: 56 - protein, 18 aa, Artificial Sequence
  source: /note=“Description of Artificial Sequence Synthetic peptide”
GSTSGSGKPGSGEGSTKG

SEQ ID NO: 57 - protein, 11 aa, Artificial Sequence
  source: /note=“Description of Artificial Sequence Synthetic peptide”
QASEDIYSLLA

SEQ ID NO: 58 - protein, 7 aa, Artificial Sequence
  source: /note=“Description of Artificial Sequence Synthetic peptide”
DASDLAS

SEQ ID NO: 59 - protein, 15 aa, Artificial Sequence
  source: /note=“Description of Artificial Sequence Synthetic peptide”
QGNYGSSSSSSYGAV

SEQ ID NO: 60 - protein, 9 aa, Artificial Sequence
  source: /note=“Description of Artificial Sequence Synthetic peptide”
IDLSSYTMG

SEQ ID NO: 61 - protein, 16 aa, Artificial Sequence
  source: /note=“Description of Artificial Sequence Synthetic peptide”
IISSGGRTYYASWAKG

SEQ ID NO: 62 - protein, 12 aa, Artificial Sequence
  source: /note=“Description of Artificial Sequence Synthetic peptide”
GRYTGYPYYFAL

SEQ ID NO: 63 - protein, 11 aa, Artificial Sequence
  source: /note=“Description of Artificial Sequence Synthetic peptide”
QASEAIYSLLA

SEQ ID NO: 64 - protein, 11 aa, Artificial Sequence
  source: /note=“Description of Artificial Sequence Synthetic peptide”
QASEDIASLLA

SEQ ID NO: 65 - protein, 7 aa, Artificial Sequence
  source: /note=“Description of Artificial Sequence Synthetic peptide”
AASDLAS

SEQ ID NO: 66 - protein, 15 aa, Artificial Sequence
  source: /note=“Description of Artificial Sequence Synthetic peptide”
QGAYGSSSSSSYGAV

SEQ ID NO: 67 - protein, 9 aa, Artificial Sequence
  source: /note=“Description of Artificial Sequence Synthetic peptide”
IDLSSYAMG

SEQ ID NO: 68 - protein, 16 aa, Artificial Sequence
  source: /note=“Description of Artificial Sequence Synthetic peptide”
IISSAGRTYYASWAKG

SEQ ID NO: 69 - protein, 16 aa, Artificial Sequence
  source: /note=“Description of Artificial Sequence Synthetic peptide”
IISSGGATYYASWAKG

SEQ ID NO: 70 - protein, 12 aa, Artificial Sequence
  source: /note=“Description of Artificial Sequence Synthetic peptide”
GAYTGYPYYFAL

SEQ ID NO: 71 - protein, 12 aa, Artificial Sequence
  source: /note=“Description of Artificial Sequence Synthetic peptide”
GRATGYPYYFAL